SEQUENCE LISTING (33 sequences)

SEQ ID NO: 1 - DNA, 368 bp, Artificial Sequence
  Synthetic Polynucleotide
aaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtatagaaccgatcctcccatgttttagagctagaaatagcaagttaaaataaggctagtccgttatcaacttgaaaaagtggcaccgagtcggtgcttttttt

SEQ ID NO: 2 - DNA, 381 bp, Artificial Sequence
  Synthetic Polynucleotide
aaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtacctcatcaggaacatgtgtttaagagctatgctggaaacagcagaaatagcaagtttaaataaggctagtccgttatcaacttgaaaaagtggcaccgagtcggtgcttttttt

SEQ ID NO: 3 - DNA, 490 bp, Artificial Sequence
  Synthetic Polynucleotide
ggtttaccgagctcttattggttttcaaacttcattgactgtgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaatatagaaccgatcctcccattggtatatatccaatgggaggatcggttctatacttgtggaaaggacgaaacaccgtacctcatcaggaacatgtgtttaagagctatgctggaaacagcagaaatagcaagtttaaataaggctagtccgttatcaacttgaaaaagtggcaccgagtcggtgctttttttggtgcgtttttatgcttgtagtattgtataatgttttt

SEQ ID NO: 4 - DNA, 407 bp, Artificial Sequence
  Synthetic Polynucleotide
aaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaaccaatgggaggatcggttctatatatatattatagaaccgatcctcccattggcttgtggaaaggacgaaacaccgtacctcatcaggaacatgtgtttaagagctatgctggaaacagcagaaatagcaagtttaaataaggctagtccgttatcaacttgaaaaagtggcaccgagtcggtgcttttttt

SEQ ID NO: 5 - DNA, 490 bp, Artificial Sequence
  Synthetic Polynucleotide
ggtttaccgagctcttattggttttcaaacttcattgactgtgccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaatatagaaccgatcctcccattggtatatattatagaaccgatcctcccattggcttgtggaaaggacgaaacaccgtacctcatcaggaacatgtgtttaagagctatgctggaaacagcagaaatagcaagtttaaataaggctagtccgttatcaacttgaaaaagtggcaccgagtcggtgctttttttggtgcgtttttatgcttgtagtattgtataatgttttt

SEQ ID NO: 6 - DNA, 814 bp, Artificial Sequence
  Synthetic Polynucleotide
tctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcaaaaccttcatcaaccacacccagggcatccccgacttctttaagcagtccttccctgaggtaagtggtcctacctcatcaggaacatgtgttttagagctagaaatagcaagttaaaataaggctagtccgttatcaacttgaaaaagtggcaccgagtcggtgctactaactctcgagtcttctttttttttttcacagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagatgctgtaccccgctgacggcggcctggaaggcagaagcgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacctacgtcgagcagcacgaggtggctgtggccagatactgcg

SEQ ID NO: 7 - DNA, 854 bp, Artificial Sequence
  Synthetic Polynucleotide
atggtgtctaagggcgaagagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcaaaaccttcatcaaccacacccagggcatccccgacttctttaagcagtccttccctgaggtaagtggtcctatagaaccgatcctcccatgttttagagctagaaatagcaagttaaaataaggctagtccgttatcaacttgaaaaagtggcaccgagtcggtgctactaactcgagtcttctttttttttttcacagggcttcacatgggagagagtcaccacatacgaagacgggggcgtgctgaccgctacccaggacaccagcctccaggacggctgcctcatctacaacgtcaagatcagaggggtgaacttcccatccaacggccctgtgatgcagaagaaaacactcggctgggaggcctccaccgagatgctgtaccccgctgacggcggcctggaaggcagaagcgacatggccctgaagctcgtgggcgggggccacctgatctgcaacttgaagaccacatacagatccaagaaacccgctaagaacctcaagatgcccggcgtctactatgtggacagaagactggaaagaatcaaggaggccgacaaagagacctacgtcgagcagcacgaggtggctgtggccagatactgcgacctccctagcaaactggggcacaaacttaattga

SEQ ID NO: 8 - DNA, 284 bp, Artificial Sequence
  Synthetic Polynucleotide
gctccgaatttctcgacagatctcatgtgattacgccaagctacgggcggagtactgtcctccgagcggagtactgtcctccgagcggagtactgtcctccgagcggagtactgtcctccgagcggagttctgtcctccgagcggagactctagatatagaaccgatcctcccattggaattctaggcgtgtacggtgggaggcctatataagcagagctcgtttagtgaaccgtcagatcgcctcgagtatagaaccgatcctcccattggatccaattcgac

SEQ ID NO: 9 - DNA, 283 bp, Artificial Sequence
  Synthetic Polynucleotide
gctccgaatttctcgacagatctcatgtgattacgccaagctacgggcggagtactgtcctccgagcggagtactgtcctccgagcggagtactgtcctccgagcggagtactgtcctccgagcggagttctgtcctccgagcggagactctagatacctcatcaggaacatgttggaattctaggcgtgtacggtgggaggcctatataagcagagctcgtttagtgaaccgtcagatcgcctcgagtacctcatcaggaacatgttggatccaattcgacc

SEQ ID NO: 10 - DNA, 1093 bp, Artificial Sequence
  Synthetic Polynucleotide
atggctgaaggatccgtcgccaggcagcctgacctcttgacctgcgacgatgagccgatccatatccccggtgccatccaaccgcatggactgctgctcgccctcgccgccgacatgacgatcgttgccggcagcgacaaccttcccgaactcaccggactggcgatcggcgccctgatcggccgctctgcggccgatgtcttcgactcggagacgcacaaccgtctgacgatcgccttggccgagcccggggcggccgtcggagcaccgatcactgtcggcttcacgatgcgaaaggtaagtggtcctatagaaccgatcctcccatgttttagagctagaaatagcaagttaaaataaggctagtccgttatcaacttgaaaaagtggcaccgagtcggtgctactaactctcgagtcttctttttttttttcacaggacgcaggcttcatcggctcctggcatcgccatgatcagctcatcttcctcgagctcgagcctccccagcgggacgtcgccgagccgcaggcgttcttccgccgcaccaacagcgccatccgccgcctgcaggccgccgaaaccttggaaagcgcctgcgccgccgcggcgcaagaggtgcggaagattaccggcttcgatcgggtgatgatctatcgcttcgcctccgacttcagcggcgaagtgatcgcagaggatcggtgcgccgaggtcgagtcaaaactaggcctgcactatcctgcctcaaccgtgccggcgcaggcccgtcggctctataccatcaacccggtacggatcattcccgatatcaattatcggccggtgccggtcaccccagacctcaatccggtcaccgggcggccgattgatcttagcttcgccatcctgcgcagcgtctcgcccgtccatctggaattcatgcgcaacataggcatgcacggcacgatgtcgatctcgattttgcgcggcgagcgactgtggggattgatcgtttgccatcaccgaacgccgtactacgtcgatctcgatggccgccaagcctgcgagctagtcgcccaggttctggcctggcagatcggcgtgatggaagagtga

SEQ ID NO: 11 - DNA, 1092 bp, Artificial Sequence
  Synthetic Polynucleotide
atggctgaaggatccgtcgccaggcagcctgacctcttgacctgcgacgatgagccgatccatatccccggtgccatccaaccgcatggactgctgctcgccctcgccgccgacatgacgatcgttgccggcagcgacaaccttcccgaactcaccggactggcgatcggcgccctgatcggccgctctgcggccgatgtcttcgactcggagacgcacaaccgtctgacgatcgccttggccgagcccggggcggccgtcggagcaccgatcactgtcggcttcacgatgcgaaaggtaagtggtcctacctcatcaggaacatgtgttttagagctagaaatagcaagttaaaataaggctagtccgttatcaacttgaaaaagtggcaccgagtcggtgctactaactctcgagtcttctttttttttttcacaggacgcaggcttcatcggctcctggcatcgccatgatcagctcatcttcctcgagctcgagcctccccagcgggacgtcgccgagccgcaggcgttcttccgccgcaccaacagcgccatccgccgcctgcaggccgccgaaaccttggaaagcgcctgcgccgccgcggcgcaagaggtgcggaagattaccggcttcgatcgggtgatgatctatcgcttcgcctccgacttcagcggcgaagtgatcgcagaggatcggtgcgccgaggtcgagtcaaaactaggcctgcactatcctgcctcaaccgtgccggcgcaggcccgtcggctctataccatcaacccggtacggatcattcccgatatcaattatcggccggtgccggtcaccccagacctcaatccggtcaccgggcggccgattgatcttagcttcgccatcctgcgcagcgtctcgcccgtccatctggaattcatgcgcaacataggcatgcacggcacgatgtcgatctcgattttgcgcggcgagcgactgtggggattgatcgtttgccatcaccgaacgccgtactacgtcgatctcgatggccgccaagcctgcgagctagtcgcccaggttctggcctggcagatcggcgtgatggaagagtga

SEQ ID NO: 12 - DNA, 141 bp, Artificial Sequence
  Synthetic Polynucleotide
gggtagtttgcagttttaaaattatgttttaaaatggactatcatatgcttaccgtaacttgaaatatagaaccgatcctcccattggtatatatccaatgggaggatcggttctatacttgtggaaaggacgaaacaccg

SEQ ID NO: 13 - protein, 45 aa, Artificial Sequence
  Synthetic Polypeptide
GFAVLKLCFKMDYHMLTVTNIEPILPLVYIQWEDRFYTCGKDETP

SEQ ID NO: 14 - DNA, 141 bp, Artificial Sequence
  Synthetic Polynucleotide
gggtagtttgcagttttaaaattatgttttaaaatggactatcatatgcttaccgtaacttgaaaccaatgggaggatcggttctatatatatattatagaaccgatcctcccattggcttgtggaaaggacgaaacaccg

SEQ ID NO: 15 - protein, 45 aa, Artificial Sequence
  Synthetic Polypeptide
GFAVLKLCFKMDYHMLTVTNQWEDRFYIYIIEPILPLACGKDETP

SEQ ID NO: 16 - DNA, 141 bp, Artificial Sequence
  Synthetic Polynucleotide
gggtagtttgcagttttaaaattatgttttaaaatggactatcatatgcttaccgtaacttgaaatatagaaccgatcctcccattggtatatattatagaaccgatcctcccattggcttgtggaaaggacgaaacaccg

SEQ ID NO: 17 - protein, 45 aa, Artificial Sequence
  Synthetic Polypeptide
GFAVLKLCFKMDYHMLTVTNIEPILPLVYIIEPILPLACGKDETP

SEQ ID NO: 18 - DNA, 311 bp, Artificial Sequence
  Synthetic Polynucleotide
aaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaatatagaaccgatcctcccattgggtatttcgatttcttggctttatatattatagaaccgatcctcccattggcttgtggaaaggagcaaacaccg

SEQ ID NO: 19 - protein, 97 aa, Artificial Sequence
  Synthetic Polypeptide
KVGQEEGLFPMIPSYLHIRYKAVREIIRINLTVNTKILVQNTRRKFLGFAVLKLCFKMDYHMLTVTNIEPILPLGISISWLYILNRSSHWLVERTKH

SEQ ID NO: 20 - DNA, 23 bp, Artificial Sequence
  Synthetic Polynucleotide
tatagaaccgatcctcccattgg

SEQ ID NO: 21 - DNA, 22 bp, Artificial Sequence
  Synthetic Polynucleotide
tacctcatcaggaacatgttgg

SEQ ID NO: 22 - DNA, 484 bp, Artificial Sequence
  Synthetic Polynucleotide
tttctcgagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgaggtaggcgtgtacggtgggaggcctatataagcagagctcgtttagtgaaccgtcagatcgcctatagaaccgatcctcccatgttttagagctagaaatagcaagttaaaataaggctagtccgttatcaacttgaaaaagtggcaccgagtcggtgcctagtaataaaggatcctttatcttcattggatccgtgtgttggttttttgtgtgcggcccgtctag

SEQ ID NO: 23 - DNA, 76 bp, Artificial Sequence
  Synthetic Polynucleotide
gttttagagctagaaatagcaagttaaaataaggctagtccgttatcaacttgaaaaagtggcaccgagtcggtgc

SEQ ID NO: 24 - DNA, 23 bp, Artificial Sequence
  Synthetic Polynucleotide
acgtcaacgtttcgcaccatcgg

SEQ ID NO: 25 - DNA, 23 bp, Artificial Sequence
  Synthetic Polynucleotide
gcttaatacgggctaatcttggg

SEQ ID NO: 26 - DNA, 23 bp, Artificial Sequence
  Synthetic Polynucleotide
acttggctacctcgttcgacagg

SEQ ID NO: 27 - DNA, 23 bp, Artificial Sequence
  Synthetic Polynucleotide
ttggcctacgtactgctctatgg

SEQ ID NO: 28 - DNA, 23 bp, Artificial Sequence
  Synthetic Polynucleotide
actagctatagattatcctaggg

SEQ ID NO: 29 - DNA, 20 bp, Artificial Sequence
  Synthetic Polynucleotide
acgtcaacgtttcgcaccat

SEQ ID NO: 30 - DNA, 20 bp, Artificial Sequence
  Synthetic Polynucleotide
gcttaatacgggctaatctt

SEQ ID NO: 31 - DNA, 20 bp, Artificial Sequence
  Synthetic Polynucleotide
acttggctacctcgttcgac

SEQ ID NO: 32 - DNA, 20 bp, Artificial Sequence
  Synthetic Polynucleotide
ttggcctacgtactgctcta

SEQ ID NO: 33 - DNA, 20 bp, Artificial Sequence
  Synthetic Polynucleotide
actagctatagattatccta

(19) United States
(12) Patent Application Publication
(10) Pub. No.: US 2016/0326546 A1
(43) Pub. Date: Nov. 10, 2016

(54) GENERATION OF LAYERED TRANSCRIPTIONAL CIRCUITRY USING CRISPR SYSTEMS

(71) Applicants: Massachusetts Institute of Technology, Cambridge, MA (US); Raytheon BBN Technologies, Corp., Cambridge, MA (US)

(72) Inventors: Samira Kiani, Somerville, MA (US); Ron Weiss, Newton, MA (US); Jacob S. Beal, Somerville, MA (US); Mohammad Reza Ebrahimkhani, Somerville, MA (US); Zhen Xie, Beijing (CN); Yinqing Li, Cambridge, MA (US)

(73) Assignees: Massachusetts Institute of Technology, Cambridge, MA (US); Raytheon BBN Technologies, Corp., Cambridge, MA (US)

(21) Appl. No.: 15/146,653

(22) Filed: May 4, 2016

Related U.S. Application Data
(60) Provisional application No. 62/156,555, filed on May 4, 2015.

(51) Int. Cl. C12N 15/85

(52) U.S. Cl. CPC C12N 15/85; C12N 2830/002

(57) ABSTRACT

Provided herein, in some embodiments, are modular transcriptional architectures and methods for regulated expression of guide RNAs in cells, such as human cells, which are based on Clustered Regularly Interspaced Palindromic Repeats (CRISPR) systems.

### RELATED APPLICATIONS

This application claims the benefit under 35 U.S.C. §119(e) from U.S. provisional application No. 62/156,555, filed May 4, 2015, which is incorporated by reference herein in its entirety.

### FEDERALLY SPONSORED RESEARCH

This invention was made with Government support under Grant Nos. P50 GM098792 and R01 CA155320 awarded by the National Institutes of Health and under Grant No. W911NF-11-2-0054 awarded by the Army Research Office. The Government has certain rights in the invention.

### BACKGROUND

Engineered biological circuits provide insight into the underlying biology of living cells and offer potential solutions to a range of medical and industrial challenges. A prerequisite for efficient engineering of such sophisticated circuits is the availability of a library of regulatory devices that can be connected in various contexts to create new and predictable behaviors. In synthetic biology, a regulatory device is typically a set of biochemical regulatory interactions that implements a basic information-processing relationship between inputs and outputs.

### SUMMARY

Provided herein, in some embodiments, are modular transcriptional modulation (e.g., repression) architectures and methods for regulating expression of guide RNAs in cells, such as human cells. These architectures and methods are based, in part, on Clustered Regularly Interspaced Palindromic Repeats (CRISPR) systems. The CRISPR regulatory devices of the present disclosure can be layered (e.g., in human cells) to create functional cascaded circuits, providing a valuable toolbox for bioengineering and other biotechnological applications.

For the transcriptional devices as provided herein, the input is typically expression of a gene product that regulates production of output from a corresponding promoter. For example, repressor devices and activator devices can be used to build computational circuits.

To date, an impediment to engineering larger and more complex circuits in any living organism is the lack of an efficient framework for generating a sufficient number of ‘composable’ regulatory devices (having matching input and output types and expression levels) that can interconnect to form functional circuits.

Recent efforts toward developing a transcriptional framework for a large library of composable devices include the creation of synthetic transcriptional modifiers by fusing effector domains to zinc-finger proteins or transcription activator-like effector proteins, albeit with limitations such as extensive DNA assembly protocols or slow temporal responses, because of the epigenetic modifications caused by the effector domains at target promoters. The Cas9 protein from the Streptococcus pyogenes CRISPR-Cas immune system has recently been adapted for both RNA-guided genome editing and gene regulation in a variety of organisms. This mechanism is attractive for engineering a large library of devices in mammalian cells because Cas9 can be targeted to virtually any DNA sequence by means of a small guide RNA (gRNA) and thus can be easily programmed for the generation of a diverse device library. In addition, catalytically inactive Cas9 protein (referred to as ‘dCas9’ or ‘Cas9m’), not fused to any effector domains, represses both synthetic and endogenous genes through steric blocking of transcription initiation and elongation. Therefore, the CRISPR system was used, as described herein, to generate synthetic gene regulatory devices and circuits. Specifically, expression of gRNAs was regulated in human cells using both RNA polymerase type II (RNA Pol II) and RNA polymerase type III (RNA Pol III) promoters, and it was demonstrated that CRISPR repressor devices can be layered to create functional circuits with high on/off ratios.

Two CRISPR families of promoters regulated by Cas9m-mediated steric blocking of transcription are provided: CRISPR-responsive RNA Pol II promoters (CRP; FIG. 2A-FIG. 2D) and CRISPR-responsive RNA Pol III promoters (e.g., CR-U6; FIG. 3A). Both families are modular and extensible because orthogonal and highly specific repressor-promoter pairs can be created by altering the Cas9 and guide RNA (gRNA) target sequence and corresponding gRNA sequence. Initially, the ability of CRISPR-responsive promoters (CRPs) to regulate expression of enhanced yellow fluorescent protein (EYFP) based on the presence or absence of gRNA constitutively expressed from a standard U6 promoter was tested. Flow cytometry analysis 48 hours after transfection of regulatory circuitry into human embryonic kidney 293 (HEK293) cells showed ˜100-fold repression for two different gRNA and CRP pairs (gRNA-a and gRNA-b; FIG. 4A-FIG. 4E), and minimal cross-talk between the two devices, which demonstrated the desired orthogonality (FIG. 5A-FIG. 5D).

Thus, in some embodiments, provided herein are engineered nucleic acids comprising a CRISPR-responsive promoter (CRP) comprising a transcription start site flanked by a first gRNA target site and a second gRNA target site.

Also provided herein, in some embodiments, are engineered nucleic acids comprising a RNA Pol II promoter flanked by a first gRNA target site and a second gRNA target site, wherein the RNA Pol II promoter is operably linked to a nucleic acid encoding a product.

Further provided herein, in some embodiments, are engineered nucleic acids comprising a RNA Pol III promoter comprising a sense strand, an antisense strand and a TATA sequence flanked by a first gRNA target site and a second gRNA target site is provided.

In some embodiments, the present disclosure provides cells comprising any of one or a combination of engineered nucleic acids described above is disclosed.

In some embodiments, the disclosure provides libraries comprising pairs of engineered nucleic acids, wherein each pair comprises (a) an engineered nucleic acid comprising a CRISPR-responsive promoter comprising a transcription start site flanked by a first gRNA target site and a second gRNA target site, and (b) an engineered nucleic acid comprising a nucleic acid encoding a gRNA, wherein the gRNA binds to the first and second gRNA target sites of the engineered nucleic acid of (a).

In some embodiments, a cell comprises (a) an engineered nucleic acid comprising a response element located upstream from a CRISPR-responsive promoter comprising a transcription start site flanked by a first gRNA target site and a second gRNA target site, wherein the promoter is operably linked to a detectable molecule, (b) an engineered nucleic acid comprising a CRISPR-responsive promoter comprising a transcription start site flanked by a third gRNA target site and a fourth gRNA target site, wherein the promoter is operably linked to a nucleic acid encoding a gRNA that binds to the first and second gRNA target sites of the engineered nucleic acid of (a), and (c) an engineered nucleic acid comprising a promoter operably linked to a nucleic acid encoding a gRNA that binds to the third and fourth gRNA target sites of the engineered nucleic acid of (b).

### BRIEF DESCRIPTION OF THE DRAWINGS

The following drawings form part of and are included to further demonstrate certain aspects o, the present disclosure, which can be better understood by reference to one or more of these drawings in combination with the detailed description of specific embodiments presented herein. The data illustrated in the drawings in no way limit the scope of the disclosure.

FIGS. 1A-IC describe examples of CRISPR repressible promoter (CRP) architectures and associated data.

FIGS. 2A-2D show proposed mechanisms of an example of a CRISPR-based transcriptional repression device.

FIGS. 3A-3B show examples of CR-U6 architectures, sequences and associated data. The sequences in FIG. 3A, from top to bottom, correspond to SEQ ID NOs: 12-17. The sequences in FIG. 3B correspond to SEQ ID NOs: 18-19.

FIGS. 4A-4E show an example of a CRISPR repression device based on a standard U6 promoter used in human cells and associated data. The sequences in FIG. 4A, from top to bottom, correspond to SEQ ID NOs: 20-21.

FIGS. 5A-5D show an analysis of the cross-talk between gRNAs and CRP-a or CRP-b.

FIGS. 6A-6D show the design and experimental analysis in human cells of CRISPR repression devices and circuits based on the RNA Pol III U6 promoter.

FIGS. 7A-7B show the output EYFP as a function of constitutive fluorescent protein for U6/CRa-U6/CRP-b cascade.

FIGS. 8A-8B show the design and function of direct gRNA-a regulation by TRE promoter, an RNA Pol II promoter. The sequence corresponds to SEQ ID NO: 22.

FIGS. 9A-9D show the design and experimental analysis in human cells of CRISPR repression devices and circuits based on RNA Pol II promoters.

FIGS. 10A-10D show the design and function of an example of an intronic gRNA-(igRNA) based repression device. The sequence in FIG. 10B corresponds to SEQ ID NO: 23,

FIGS. 11A-11B show data demonstrating reversibility of inducible CRISPR transcriptional repression.

FIGS. 12A-12B show that repression of output in an example intronic gRNA device is not due to overexpression of mKate protein and load on host cellular resources.

FIGS. 13A-13C show Cas9m and igRNA-a repression is reduced by modification of an intronic branch point sequence.

FIG. 14 shows an igRNA device library depicting the nucleotide sequence of target sites at CRP promoters and corresponding guide sequence on igRNA.

FIGS. 15A-15C show an example of a cascade circuit based on layering U6-driven gRNA-a and CRP-driven gRNA-b.

FIGS. 16A-16C show an example of a cascade circuit based on layering U6-driven gRNA-b and CRP-B/igRNA-a.

FIGS. 17A-17B show three cascades of transcriptional repression devices with stage 1 gRNAs expressed from RNA Pol II promoters.

FIGS. 18A-18B show an example distribution of constitutive fluorescence.

FIGS. 19A-19B show a 7-AAD viability test following transfection with an igRNA-a device.

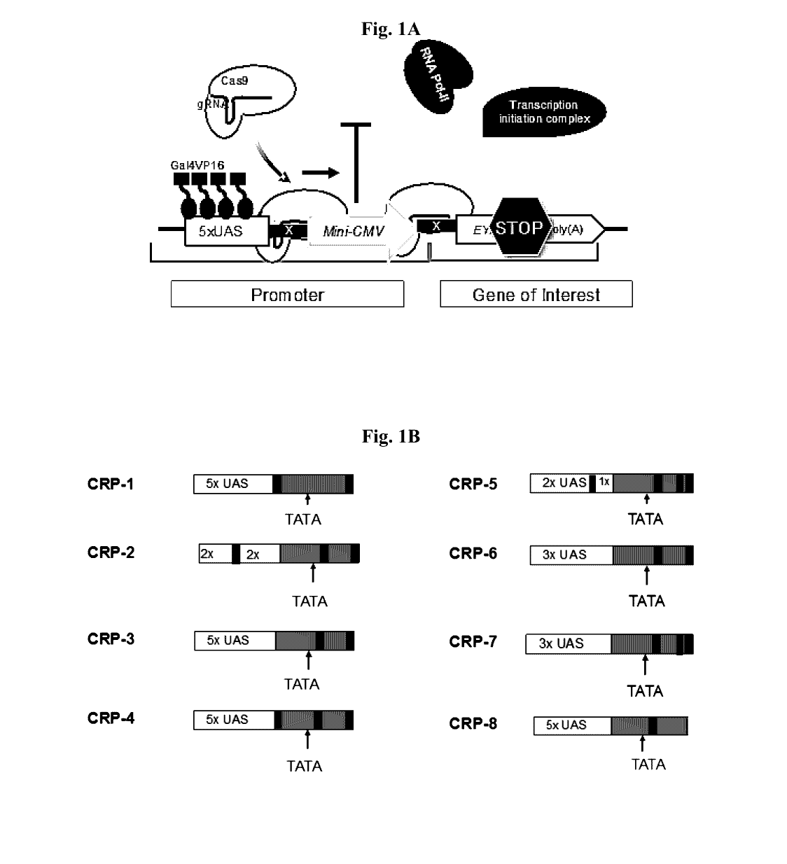

### DETAILED DESCRIPTION

Provided herein, in some embodiments, are regulatory devices that comprise a set of biochemical regulatory interactions between input and output that implement the repression of transcription of a nucleic acid sequence encoding a product. Such regulatory devices can be used for numerous applications and are particularly useful in human cells. For example, such repressor devices can be used to control the transcription of a therapeutic compound, or a detectable molecule, or to coordinate step-wise programming or reprogramming of cells for differentiation. In some embodiments, the disclosed regulatory devices are used to deliver programs to cancerous cells that result in cell death based on the sensing intracellular molecules that correlate with diseased state or cancer. In some embodiments, the disclosed regulatory devices are used to control the expression of an antiviral gene based on the sensing of viral proteins or cellular proteins, the expression of which is dampened by viral infection. Other applications are encompassed by the present disclosure.

### Transcriptional Repression Devices

Transcriptional repression devices, as provided herein, typically include an engineered nucleic acid that comprises a CRISPR-responsive promoter (CRP), which includes comprising a transcription start site flanked by a first gRNA target site and a second gRNA target site.

### CRISPR-Responsive Promoters

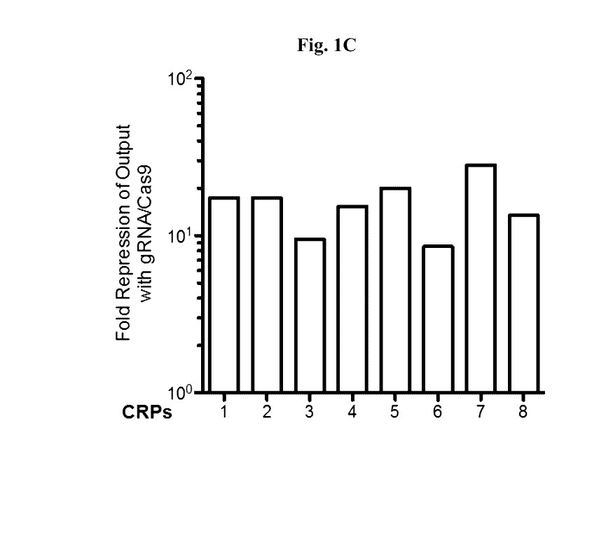

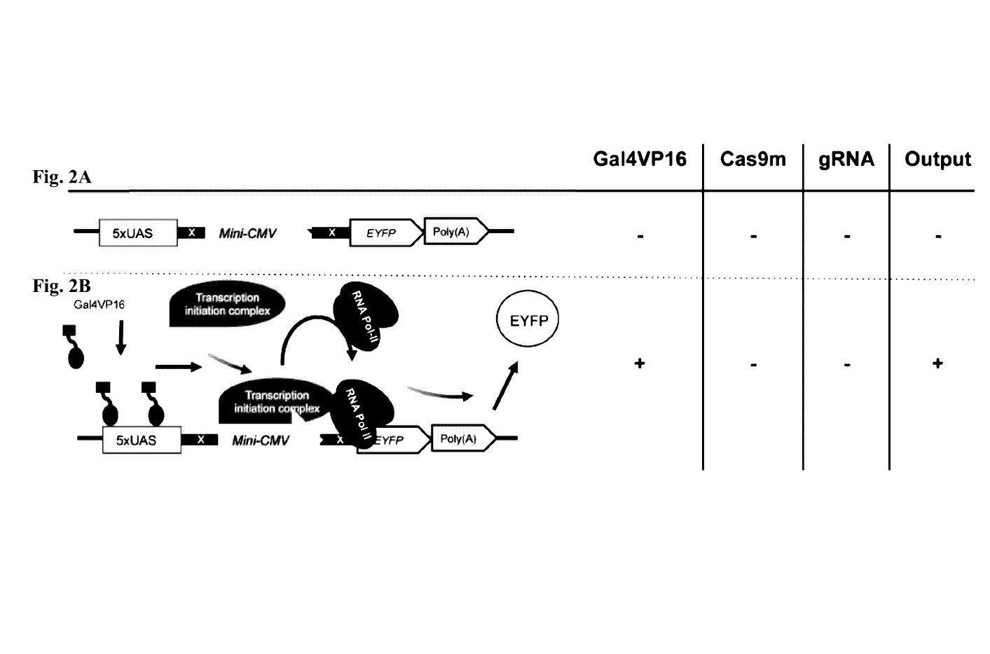

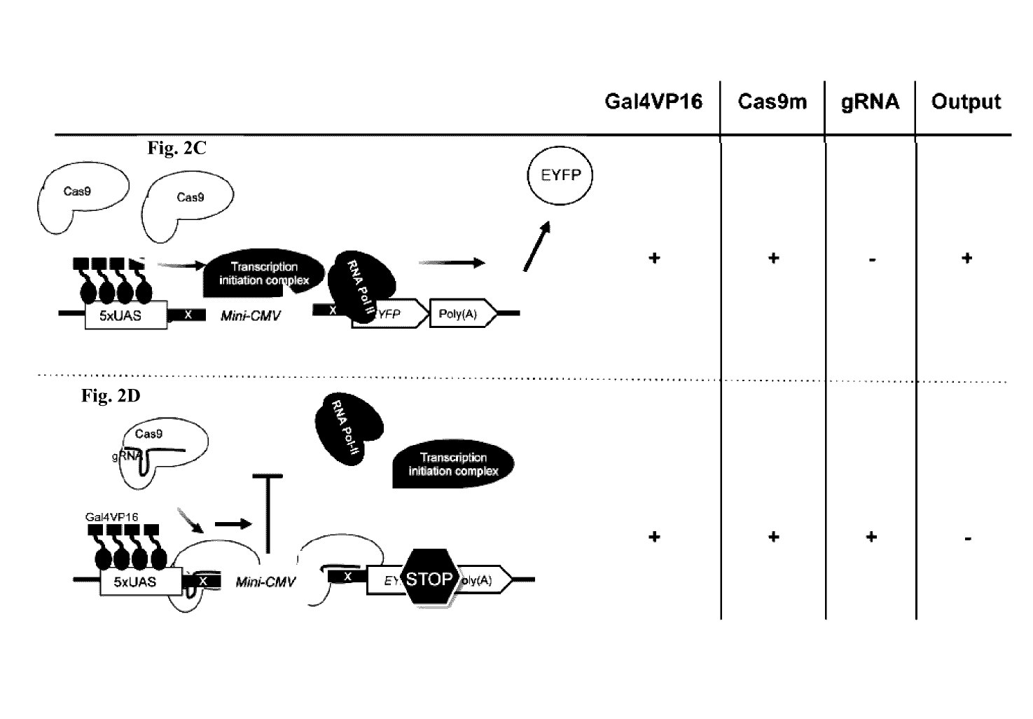

A CRISPR-responsive promoters (CRP) is a promoter regulated (e.g., repressed or activated) by Cas9. In some embodiments, the Cas9 is catalytically inactive Cas9m. Without being bound by theory, catalytically-inactive Cas9m represses transcription through a steric blocking mechanism (see, e.g., FIGS. 2A-2D). Generally, Cas9m is guided by a guide RNA that binds specifically to target sites flanking a minimal promoter, such that a transcription initiation complex is blocked (sterically hindered) from binding to the promoter to initiate transcription of a particular gene or other product/output.

CRISPR-responsive promoters of the present disclosure are typically eukaryotic promotors, which are particularly useful in human cells. To date, synthetic gene regulatory devices and circuits for regulated expression in human cells has been limited. The present disclosure provides a ‘toolbox’ to facilitate a wide range of applications in human cells, for example.

Thus, in some embodiments, a CRISPR-responsive promoter comprises a RNA Pol II promoter or an RNA Pol II promoter.

A promoter, generally, is a region of nucleic acid that initiates transcription of a nucleic acid encoding a product. A promoter may be located upstream (e.g., 0 bp to −100 bp, −30 bp, −75 bp, or −90 bp) from the transcriptional start site of a nucleic acid encoding a product, or a transcription start site may be located within a promoter. A promoter may have a length of 100-1000 nucleotide base pairs, or 50-2000 nucleotide base pairs. In some embodiments, promoters have a length of at least 2 kilobases (e.g., 2-5 kb, 2-4 kb, or 2-3 kb).

In some embodiments, a promoter is an RNA pol II promoter. RNA pol II promoters comprise a DNA sequence that is sufficient to direct accurate initiation of transcription of the nucleic acid encoding a product that is located downstream of the promoter by RNA polymerase II machinery, or a minimal essential promoter. In some embodiments, a RNA pol II promoter is a mini-CMV promoter. Other RNA pol II promoters (e.g., CMV promoter) are encompassed by the present disclosure.

Thus, in some aspects, the disclosure provides an engineered nucleic acid comprising a RNA Pol II promoter flanked by a first gRNA target site and second gRNA target site, wherein the RNA Pol II promoter is operably linked to a nucleic acid encoding a product. In some embodiments, the RNA Pol II promoter is a mini-CMV promoter. In some embodiments, an engineered nucleic acid comprising a RNA Pol II promoter flanked by a first gRNA target site and second gRNA target site comprises a response element located upstream from the first gRNA target site. In some embodiments, the response element is at least one UAS site (e.g., 1×UAS, 2×UAS, 4×UAS, 5×UAS).

In some embodiments, a promoter is an RNA pol Ill promoter. In some embodiments, the RNA pol III promoter is a RNA pol III U6 (or U6) promoter. RNA Pol II promoters, such as U6, in some embodiments, are useful for the expression of small RNAs, including small interfering RNA, short hairpin RNA, and guide RNA, for CRISPR editing systems. Other RNA pol III promoters (e.g., H1) are encompassed by the present disclosure.

In some aspects, the disclosure provides an engineered nucleic acid comprising a RNA Pol III promoter comprising a sense strand, an antisense strand and a TSS flanked by a first gRNA target site and a second gRNA target site. In some embodiments, the TSS is TATA sequence. In some embodiments, the RNA Pol III promoter is a U6 promoter. In some embodiments, an engineered nucleic acid comprising a RNA Pol III promoter comprising a sense strand, an antisense strand and a TSS flanked by a first gRNA target site and a second gRNA target site further comprises a response element located upstream from the first gRNA target site. In some embodiments, the first gRNA target site and the TSS are separated by at least 20 nucleotide base pairs (e.g., 20, 25, 30, or 50 bp), at least 40 nucleotide base pairs (e.g., 40, 45, 50, or 100 bp), at least 60 nucleotide base pairs (e.g., 60, 80, 100, or 150 bp), at least 80 nucleotide base pairs (e.g., 80, 90, 100, 150, or 200 bp), at least 100 nucleotide base pairs (e.g., 100, 120, 150, or 200 bp), or at least 200 (e.g., 200, 250, 300, or 500 bp) nucleotide base pairs.

Any of a number of promoters suitable for use in a cell (e.g., a human cell) may be used in the transcriptional repressor devices of the present disclosure. A promoter may be, for example, a constitutive promote or an inducible promoter. In some embodiments, a promoter is a tissue-specific promoter or a developmental stage-specific promoter. Promoters, as used herein, may be naturally occurring or synthetic. In some embodiments, promoters are bidirectional, wherein the 5′ ends of two nucleic acids that each encode a product on opposite strands flank the promoter.

For example, constitutive promoters having different strengths can be used. A nucleic acid described herein may include one or more constitutive promoters, such as viral promoters or mammalian promoters (obtained from mammalian genes) that are generally active in promoting transcription. Non-limiting examples of constitutive viral promoters include the Herpes Simplex virus (HSV), thymidine kinase (TK), Rous Sarcoma Virus (RSV), Simian Virus 40 (SV40), Mouse Mammary Tumor Virus (MMTV), Ad E1A and cytomegalovirus (CMV) promoters. Non-limiting examples of constitutive mammalian promoters include housekeeping gene promoters such β-actin promoter (e.g., chicken 3-actin promoter) and human elongation factor-1α (EF-1α) promoter.

Inducible promoters may also be used. Non-limiting examples of suitable inducible promoters include those from genes such as cytochrome P450 genes, heat shock protein genes, metallothionein genes, and hormone-inducible genes, such as the estrogen gene promoter. Another example of an inducible promoter is the tetVP16 promoter that is responsive to tetracycline.

Tissue-specific promoters and/or regulatory elements may also be used. Non-limiting examples of such promoters that may be used include hematopoietic stem cell-specific promoters.

Synthetic promoters may also be used. A synthetic promoter may comprise, for example, regions of known promoters, regulatory elements, transcription factor binding sites, enhancer elements and/or repressor elements.

It is to be understood, that any of a number of known promoters (e.g., CMV, mini-CMV, EFiα, sv40, PGK1, Ubc, human beta actin, chicken beta actin, CAG, TRE, UAS, AcS, polyhedron, CaMKIIα, GAL1, GAL10, TEF1, GDS, ADH1, CaMV35S, Ubi, H1, or U6) can be flanked by a first guide RNA (gRNA) target site and a second gRNA target site to create a CRP.

A CRP, in some embodiments, comprises a transcription start site (TSS) flanked by a first guide RNA (gRNA) target site and a second gRNA target site. In some embodiments, an entire promoter that includes a TSS is flanked by a first guide RNA (gRNA) target site and a second gRNA target site, while in other embodiments, only a portion of a promoter is flanked by a first gRNA target site and a second gRNA target site. For example, only the TSS (e.g., a TATA box/sequence) located within a promoter (or located upstream from a promoter) may be flanked by a first gRNA target site and a second gRNA target site.

In some embodiments a nucleic acid comprising a CRISPR-responsive promoter comprising a transcriptional start site flanked by a first and second gRNA target sites further comprises a response element located upstream from the first gRNA target site. A response element is a sequence of nucleotides to which a protein (e.g., transcription factor or transcription initiation complex) binds, resulting in transcriptional regulation (e.g., initiation or activation of transcription). For example, transcription activator proteins often bind to response elements upstream of a promoter to activate transcription. In some embodiments, promoters, together with other control elements, control the level of transcription of a nucleic acid encoding a product. Promoters, in some embodiments, include multiple response elements that may be identical to each other (e.g., have identical sequences) or different from each other.

### Transcriptional Start Sites

In some embodiments, nucleic acids disclosed herein comprise a transcription start site. A transcription start site is the location where transcription starts at the 5′-end of a nucleic acid encoding a product. In some embodiments, the transcription start site is located within a promoter. In some embodiments, the first gRNA target site and the TSS are separated by at least 20 nucleotide base pairs (e.g., 20, 25, 30, or 50 bp), at least 40 nucleotide base pairs (e.g., 40, 45, 50, or 100 bp), at least 60 nucleotide base pairs (e.g., 60, 80, 100, or 150 bp), at least 80 nucleotide base pairs (e.g., 80, 90, 100, 150, or 200 bp), at least 100 nucleotide base pairs (e.g., 100, 120, 150, or 200 bp), or at least 200 (e.g., 200, 250, 300, or 500 bp) nucleotide base pairs.

In some embodiments, the transcription start site is a TATA sequence (consensus sequence TATAAA), which is recognized by the general transcription factor TATA-binding protein (TBP). Many transcription start sites are known in the art and can be found in a database of transcription start sites (Suzuki, Y., et al., DBTSS, DataBase of Transcriptional Start Sites: progress report 2004, Nucleic Acids Res., 1; 32: D78-81, 2004), and are herein incorporate by reference in its entirety.

Guide RNA (gRNA) Target Site and gRNAs

A gRNA is a synthetic RNA composed of a scaffold sequence, which is necessary for Cas9- or Cas9m-binding, and a user-defined targeting sequence, which defines the complementing gRNA target site that flanks a nucleic acid encoding a product. In some embodiments, a gRNA is operably linked to a constitutive promoter. In some embodiments, a gRNA is operably linked to a CRP. For example, SEQ ID NO: 22 provides a sequence of a nucleic acid comprising a gRNA operably linked to an RNA Pol II promoter.

In some embodiments, a gRNA has length of 10-50 nucleotides. For example, a gRNA may have length of 10-20, 10-30, 10-40, 15-20, 15-20, 15-40, 15-50, 20-30, 20-40 or 20-50 nucleotides. In some embodiments, a gRNA has a length of 15, 16, 17, 18, 19, 20, 21, 2, 23, 24, 25, 26, 27, 28, 29 or 30 nucleotides. In some embodiments, a gRNA has a length of 20 nucleotides.

A guide RNA (gRNA) target site is a nucleotide sequence to which a gRNA binds. A gRNA comprises a nucleotide sequence that is complementary to (partially or wholly complementary to) a gRNA target site. A gRNA target site also comprises a Protospacer Adjacent Motif (PAM) located immediately downstream from the target site. Examples of PAM sequence are known (see, e.g., Shah S A et al. RNA Biology 10 (5): 891-899, 2013). In some embodiments, the sequence of PAM is dependent upon the species of Cas9 used in the architecture. In some embodiments, a PAM sequence is selected from the group consisting of: CGG, GGG, AGG, and TGG. Non-limiting examples of gRNA and gRNA target sequences are represented by SEQ ID NOs: 20-21, and SEQ ID NOs: 29-33. SEQ ID NOs: 24-28 represent non-limiting examples of gRNA target sequences containing a PAM sequence.

In some embodiments, the first gRNA target site is located in the sense strand upstream of the transcription start site and the second gRNA target site is located in the antisense strand downstream of the transcription start site in the engineered nucleic acid. An example sequence of a sense-antisense configuration of a CR-U6 is provided in SEQ ID NO: 3 and 12. An example amino acid sequence of a sense-antisense configuration of a CR-U6 is provided by SEQ ID NO: 13.

In some embodiments, the first gRNA target site is located in the antisense strand upstream of the transcription start site and the second gRNA target site is located in the sense strand downstream of the transcription start site in the engineered nucleic acid. An example sequence of an antisense-sense configuration of a CR-U6 is provided in SEQ ID NO: 4 and 14. An example amino acid sequence of an antisense-sense configuration of a CR-U6 is provided by SEQ ID NO: 15.

In some embodiments, the first gRNA target site is located in the sense strand upstream of the transcription start site and the second gRNA target site is located in the sense strand downstream of the transcription start site in the engineered nucleic acid. An example sequence of a sense-sense configuration of a CR-U6 is provided in SEQ ID NO: 5 and 16. An example amino acid sequence of a sense-sense configuration of a CR-U6 is provided by SEQ ID NO: 17.

In some embodiments, the first gRNA target site is located in the antisense strand upstream of the transcription start site and the second gRNA target site is located in the antisense strand downstream of the transcription start site in the engineered nucleic acid. Examples of U6-gRNA sequences are provided by SEQ ID NO: 1 and SEQ ID NO: 2. Example CRP promoter sequences are provided by SEQ ID NO: 8 and SEQ ID NO: 9.

CRPs regulate transcription of nucleic acid encoding a product based on the presence or absence of gRNA. In some embodiments, gRNA is constitutively expressed. In some embodiments, gRNA is constitutively expressed from a standard U6 promoter. In some embodiments, gRNA expression is controlled by another CRP.

### Response Elements

A response element is a sequence of nucleotides within a promoter region of a nucleic acid to which specific transcription factors bind to regulate (e.g., activate) transcription of the nucleic acid encoding a product.

In some embodiments, a nucleic acid encoding a product is controlled by UAS to which GAL4VP16 binds. In some embodiments, a tetracycline response element, to which tetracycline binds, controls the transcription of a nucleic acid encoding a product. Many response elements are known in the art and can be included in the disclosed nucleic acids or circuits (e.g., HRE (Gao S., et al., Toxicol Sci. 2013, 132(2): 379-389); p 53 (Wang B. et al., Cell Cycle. 2010, 9(5):870-9; IARC TP53 database, World Health Organization); C/EBP (Osada S., et al, J. Biol. Chem. 1996, 271: 3891-3896; Van der Sanden M., et al., J. Biol. Chem. 2004, 279:52007-52015), and CRE (koyanaqi S. et al., J. Biol. Chem. 2011, 286: 32416-23)).

It is to be understood that any number of repeats (e.g., 2-20, 2-15, 2-10, 2-5) of a response element sequence may be used to control the transcription of a nucleic acid encoding a product. For example, 2×, 3×, 4×, 5×, 6×, 7×, or 14× of a response element sequence can be designed to control transcription of a nucleic acid encoding a product. In some embodiments, the response element is located upstream from the first gRNA target site flanking a promoter or transcription start site. In some embodiments, the response element is located downstream from the first gRNA target site that flanks a promoter.

### Gene Products

In some embodiments, a product encoded by a nucleic acid is a detectable molecule. A detectable molecule is a molecule that can be visualized (e.g., using a naked eye or under a microscope). In some embodiments, the detectable molecule is a fluorescent molecule, a bioluminescent molecule, or a molecule that provides color (e.g., β-galactosidase, β-lactamasses, β-glucuronidase and spheriodenone). In some embodiments, the detectable molecule is a fluorescent protein or functional peptide or functional polypeptide thereof. The fluorescent protein may be a blue fluorescent protein, cyan fluorescent protein, green fluorescent protein, yellow fluorescent protein, orange fluorescent protein or red fluorescent protein. The blue fluorescent protein may be azurite, EBFP, EBFP2, mTagBFP or Y66H. The cyan fluorescent protein may be ECFP, AmCyan1, Cerulean, CyPet, mECFP, Midori-ishi Cyan, mTFP1 or TagCFP. The Green fluorescent protein may be AcGFP, Azami Green, EGFP, Emarald, GFP or a mutated form of GFP (e.g., GFP-S65T, mWasabi, Stemmer, Superfolder GFP, TagGFP, TurboGFP or ZsGreen). The yellow fluorescent protein may be EYFP, mBanana, mCitrine, PhiYFp, TagYFP, Topaz, Venus, YPet or ZsYellow1. Orange fluorescent protein may be DsRed, RFP, DsRed2, DsRed-Express, Ds-Red-monomer, Tomato, tdTomato, Kusabira Orange, mKO2, mOrange, mOrange2, mTangerine, TagRFP or TagRFP-T. Red fluorescent protein may be AQ142, AsRed2, dKeima-Tandem, HcRed1, tHcRed, Jred, mApple, mCherry, mPlum, mRasberry, mRFP1, mRuby or mStrawberry. In some embodiments, the fluorescent protein is mKate.

In some embodiments, the detectable molecule is EYFP. In some embodiments, the detectable molecules is mKate.

Bioluminescent molecules are bioluminescent proteins or functional polypeptides or functional peptides thereon. Examples of non-limiting bioluminescent proteins are firefly luciferase, click-beetle luciferase, and Renilla luciferase.

In some embodiments, a product encoded by a nucleic acid is a therapeutic protein, mRNA, miRNA or polypeptide. In some embodiments, the therapeutic protein, mRNA, miRNA or polypeptide is an antibody, a peptibody, a growth factor, a clotting factor, a hormone, a membrane protein, a cytokine, a chemokine, an activating or inhibitory peptide acting on a cell surface receptors or an ion channel, a thrombolytic, an enzyme, a bone morphogenetic protein, a nucleases or another protein used for gene editing, an Fc-fusion protein, or an anticoagulant.

In some embodiments, a product encoded by a nucleic acid is a differentiation-inducing factor (e.g., DIF-1, DIF-2, or DIF-3). In some embodiments, a product encoded by a nucleic acid is a transcription factor that induces cell reprogramming of cells (e.g., induced pluripotent stem cells or embryonic progenitor cells). For example, transcription factors OCT4 and SOX2 maintain pluripotency of induced pluripotent stem cells. Another non-limiting example of a differentiation inducing factor is MUC5ac, which induces the programing of tracheal epithelial cells to mucous secreting goblet cells.

In some embodiments, a product encoded by a nucleic acid is a gRNA for the control of transcription of another nucleic acid encoding a product.

In some embodiments, a product is a detectable molecule and is used as a transfection marker.

### Engineered Nucleic Acids

In some embodiments, the nucleic acid is located on (or within) a vector (e.g., a plasmid, a bacteriophage, a cosmid, or a viral vector). In some embodiments, plasmids are cloning plasmids. In some embodiments, plasmids are expression plasmids. A non-limiting example of a plasmid is pCR2.1-TOPO TA vector. In some embodiments, the engineered nucleic acid comprised by the vector is integrated into the genome of the cell, organ, organoid, or organism to which it is introduced. In some embodiments, the vector is episomal.

In some embodiments, an engineered nucleic acid is a nucleic acid that has been designed and made using known in vitro techniques in the art. In some embodiments, an engineered nucleic acid, also referred to as a circuit herein, is a nucleic acid that is not isolated from the genome of an organism. In some embodiments, the engineered nucleic acid is introduced to a cell, plurality of cells, an organ or an organism to perform diverse functions (e.g., differentiation of cells, as sensors within cells, program a cell to act as a sensor, and delivery of selective cell-based therapies). In some embodiments, the engineered nucleic acid comprises one or more nucleic acid encoding a product and control elements. Non-limiting examples of control elements include promoters, activators, repressor elements, insulators, silencers, response elements, introns, enhancers, transcriptional start sites, termination signals, linkers and poly(A) tails. Any combination of such control elements is contemplated herein (e.g., a promoter and an enhancer).

CRISPR-based devices of the present disclosure are particularly useful, for example, for scaling to large, sophisticated circuits, as the nucleic acid required for each additional device is small. This means that complex circuits can be encoded when there are size limits on the nucleic acid (e.g., DNA) to be delivered, and that any given circuit topology can be encoded in a much smaller amount of DNA. Regulation by RNA interference integrates well with CRISPR-based devices, providing a useful means of sensing and effecting cell state. Additional scaling of the CRISPR technology and the creation of more complex logic also benefits from using multiple orthogonal Cas9 proteins. Give the simplicity of creating additional gRNAs and corresponding promoters, and the high performance of the devices presented here, it is possible to rapidly generate and characterize a large library of effective regulatory devices. Taken together, the scalability and ability to rapidly design the devices as provided herein allows CRISPR based circuits to facilitate a wide range of applications in human cells.

### Layered Circuits

Biological circuits that comprise a nucleic acid encoding a product and at least one control elements (e.g., promoters, activators, repressor elements, insulators, silencers, response elements, introns, enhancers, transcriptional start sites, termination signals or poly(A) tails) enable the manipulation of cells for different purposes. For example, a biological circuit can be used to achieve the simple task of delivering a molecule to a cell that changes its differentiation state, inhibits or enhances a signaling pathway, or changes its growth rate.

However, for performing more sophisticated tasks, such as delivering a molecule based on the result of a sensing of the same or another molecule (e.g., an intracellular molecule or an extracellular molecule that alleviates disease), layering of biological circuits useful. Layered circuitry refers to the ability to compose complex multi-level control or regulatory operations with biological circuits. The difficulty is assembling components that do not interfere with each other. Provided herein are architectures/constructs and methods for layering circuits comprising components that interact with each other. Such interactions can be used to perform sophisticated tasks (e.g., step-wise differentiation of cell, or engineering sophisticated control over cell function in cell-based therapies). A sophisticated task is one that involves both an input and an output, rather than only an output. Disclosed herein, in some embodiments, are layered circuits for complex multi-level control or regulatory operations by interconnecting CRISPR-based devices.

Accordingly, in some aspects, the disclosure provides a cell (e.g., a human cell) comprising multiple circuits that interact with each other (layered circuits).

In some aspects, the disclosure provides a cell (e.g., a human cell) comprising an engineered nucleic acid comprising a CRISPR-responsive promoter (e.g., RNA Pol II such as mini-CMV or RNA Pol III such as U6) comprising a transcription start site flanked by a first gRNA target site and a second gRNA target site, wherein the CRISPR-responsive promoter is operably linked to a nucleic acid encoding a product. In some embodiments, a cell further comprises an engineered nucleic acid comprising a promoter operably linked to a nucleic acid encoding a gRNA that binds to the first and second gRNA target sites of the engineered nucleic acid, thus providing layering of the circuit.

In some embodiments, a cell comprises a nucleic acid encoding a gRNA that is flanked by cognate intronic splice sites. In some embodiments, a cell comprises a nucleic acid encoding a gRNA that is flanked by cognate intronic splice sites and is located within a nucleic acid encoding a product. In some embodiments, the product is a detectable molecule. In some embodiments, the detectable molecule is used to monitor transfection. In some embodiments, the detectable molecule is mKate. Example sequences of nucleic acids comprising intronic gRNA flanked by mKate are provide by SEQ ID NO: 6 and SEQ ID NO: 7. SEQ ID NO: 23 provides an example amino acid sequence of gRNA intron of mKate fluorescent protein coding gene. Example sequences of nucleic acids comprising intronic gRNA flanked by RFP are provide by SEQ ID NO: 10 and SEQ ID NO: 11.

In some embodiments, a cell comprising an engineered nucleic acid comprising a CRP comprising a TSS flanked by a first gRNA target site and a second gRNA target site comprises a response element upstream of a promoter operably linked to a nucleic acid encoding a product. In some embodiments, the product is a gRNA. In some embodiments, the response element is a tetracycline response element.

In some embodiments, the disclosed cell further comprises a nucleic acid that encodes an activator of transcription (e.g., GAL4VP16).

In some embodiments, the cell disclosed herein comprises cas9. In some embodiments, cas9 is encoded from an engineered nucleic acid. In some embodiments, the cas9 is catalytically inactive case9 (cas9m). In some embodiments, transcription of nucleic acid encoding cas9m is controlled by a constitutive promoter, inducible promoter, or a tissue-specific promoter. In some embodiments, a cell comprises a nucleic acid encoding cas9m regulated by a constitutive promoter, tissue-specific promoter, or inducible promoter, and also another nucleic acid encoding cas9m that is regulated by a CRISPR-responsive promoter.

In some embodiments, a cell comprises at least two (e.g., 2, 3, 4, 5, or 10) nucleic acids disclosed herein. For example, a cell may comprise a first engineered nucleic acid comprising a promoter operably linked to a nucleic acid encoding a gRNA, and a second engineered nucleic acid comprising a CRISPR-responsive promoter comprising a TSS flanked by a first and second gRNA target sites, and that is operably linked to a nucleic acid encoding a product. In some embodiments, the gRNA of the first nucleic acid binds to the gRNA target sites of the second nucleic acid. In some embodiments for example, a cell comprises a first engineered nucleic acid comprising a promoter operably linked to a nucleic acid encoding a gRNA; a second engineered nucleic acid comprising a CRISPR-responsive promoter comprising a TSS that is flanked by first and second gRNA target sites, and that is operably linked to a nucleic acid encoding a first product; and a third engineered nucleic acid comprising a CRISPR-responsive promoter comprising a TSS that is flanked by a third and fourth gRNA target sites, and that is operably linked to a nucleic acid encoding a second product. In some embodiments, the gRNA of the first nucleic acid binds to the gRNA target sites of the second, third, or second and third nucleic acids. In some embodiments, the first product is a gRNA that binds to the gRNA target sides of the third nucleic acid. In some embodiments, the promoter of the first nucleic acid that encodes the gRNA is a CRISPR-responsive promoter and the second product is another gRNA that binds to the gRNA target sites of the first nucleic acid to provide a feedback mechanism. In some embodiments, the gRNA encoded by the first and second nucleic acid is the same. In some embodiments, the gRNA encoded by the first and third gRNA is the same. In some embodiments, the gRNA encoded by the first, second and third nucleic acids is the same. In some embodiments, the gRNA of any of the first, second or third nucleic acid is flanked by cognate intronic splice sites.

It is to be understood that a cell comprising more than three layered (or interacting) nucleic acids that comprise one or more of the following components are contemplated to interacted in any configuration (a) a promoter operably linked to a nucleic acid encoding a gRNA, (b) a CRISPR-responsive promoter that comprises a TSS that is flanked by gRNA target sites and that is operably linked to a nucleic acid that encodes a product, (c) a promoter operably linked to a nucleic acid encoding Cas9m, and (d) a promoter operably linked to nucleic acid encoding a detectable molecule, that may be used as a transfection control. In some embodiments, a component of one nucleic acid interacts with another component on the same nucleic acid. In some embodiments, a component of one nucleic acid interacts with a component of another nucleic acid. In some embodiments, a component of one nucleic acid interacts with more than one component on the same or a different nucleic acid. It is to be understood that any of the gRNA can be flanked by cognate intronic splice sites of a product. The product may be an activator or transcription.

In some embodiments, a cell comprises a nucleic acid that encodes multiple gRNAs, or multiple nucleic acids each encoding one or more gRNAs (e.g., 2, 3, 4, 5, 6, or 10 or more). Any number of gRNA and gRNA target sites pairs are contemplated to be comprised by a cell. In some embodiments, actions of the different gRNA and gRNA target sites pairs are sequential. For example, a first gRNA and gRNA sites pair may control the transcription of a nucleic acid encoding a second gRNA, which then acts on its paired gRNA target sites to control the transcription of a nucleic acid encoding a third gRNA, or another product. In some embodiments, actions of the different gRNA and gRNA target sites pairs are parallel or unrelated. For example, a first gRNA and gRNA sites pair may control the transcription of a nucleic acid encoding a first product, and a second pair of gRNA and gRNA target sites control the transcription of a nucleic acid encoding a second product.

In some embodiments, a cell comprises a nucleic acid encoding a gRNA regulated by a constitutive promoter, tissue-specific promoter, or inducible promoter, and also another nucleic acid encoding the same gRNA that is regulated by a CRISPR-responsive promoter.

In some embodiments, a cell comprises an engineered nucleic acid comprising a response element upstream of the promoter operably linked to a nuclide acid encoding a product. In some embodiments, the product is a gRNA. The response element comprised in a nucleic acid comprised by the disclosed cell can be any of the response elements described above. In some embodiments, a cell disclosed herein comprises a tetracycline response element.

Thus in some embodiments, disclosed herein is a cell, comprising (a) an engineered nucleic acid comprising a response element located upstream from a CRISPR-responsive promoter comprising a transcription start site flanked by a first gRNA target site and a second gRNA target site, wherein the promoter is operably linked to a detectable molecule; (b) an engineered nucleic acid comprising a CRISPR-responsive promoter comprising a transcription start site flanked by a third gRNA target site and a fourth gRNA target site, wherein the promoter is operably linked to a nucleic acid encoding a gRNA that binds to the first and second gRNA sites of the engineered nucleic acid of (a); and (c) an engineered nucleic acid encoding a gRNA that binds to the third and fourth gRNA target sites of the engineered nucleic acid of (b).

For example, a U6 promoter can be operably linked to a nucleic acid that encodes a first gRNA, which in turn binds to target sites flanking a nucleic acid encoding a second gRNA, which in turn binds to the target sites flanking a nucleic acid encoding a product (e.g., EYFP, a transcription activator such as Ga14VP16, or a therapeutic protein).

In some embodiments, the cell comprising (a), (b) and (c) further comprises (d) an engineered nucleic acid comprising a promoter operably linked to a nucleic acid encoding catalytically inactive Cas9 (Cas9m). In some embodiments, the cell comprising (a), (b) and (c), (a), (b), (c), and (d) comprises an engineered nucleic acid encoding a protein that binds to the response element of the engineered nucleic acid of (a). In some embodiments, the promoter of (a) is a RNA Pol II promoter (e.g., mini-CMV promoter). In some embodiments, the promoter of (a) is a RNA Pol III promoter (e.g., U6 promoter). In some embodiments, the detectable molecule is a fluorescent protein In some embodiments, the response element comprised in a cell is at least one UAS sequence. IN some embodiments, the protein that binds to the response element is Ga14 (e.g., Ga14VP16). In some embodiments, the cell is a human cell.

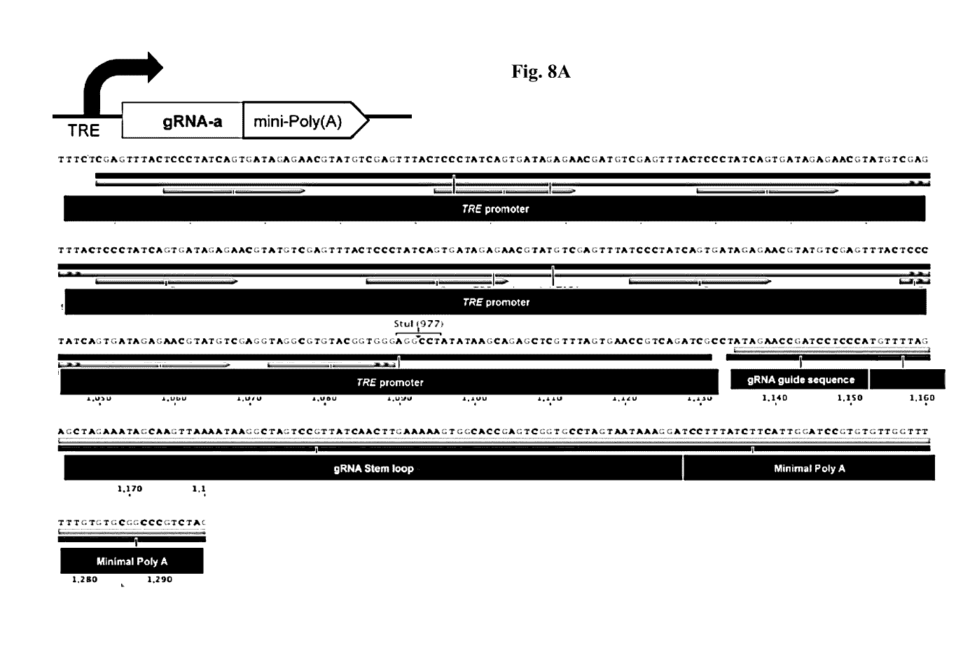

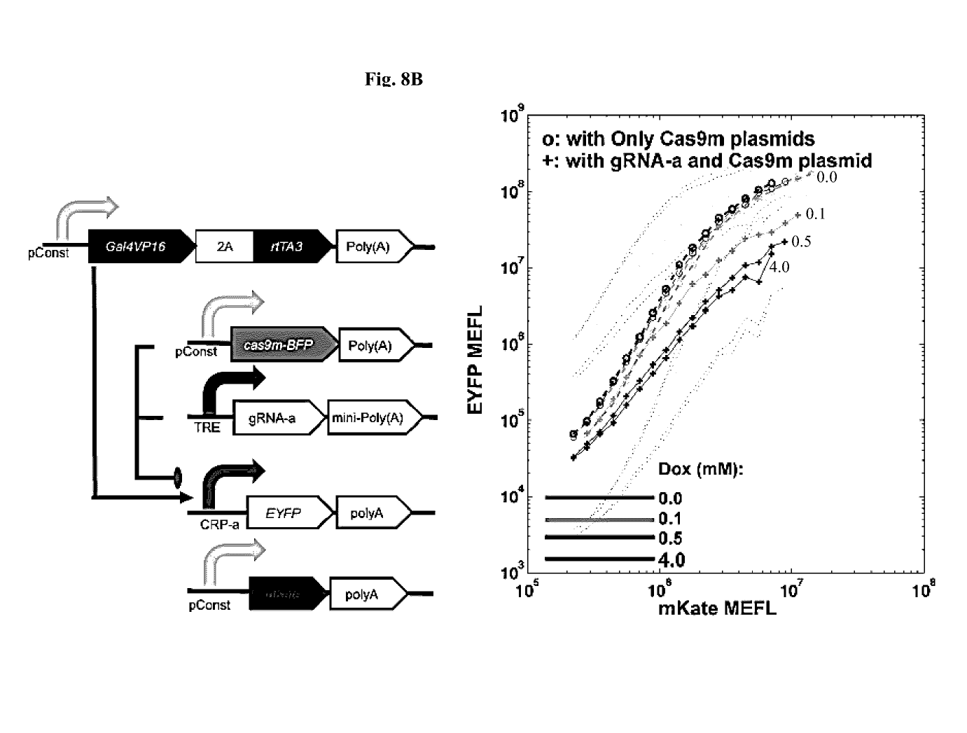

In some embodiments, a cell comprises (a) a nucleic acid comprising a constitutive promoter operably linked to a transcription activator (e.g., Ga14VP16) that activates the transcription of (b) a nucleic acid encoding a product (e.g., EYFP), which is operably linked to a promoter comprising a TSS that is flanked by gRNA target sites corresponding to (c) a gRNA that is operably linked to a promoter comprising a response element (e.g., TRE). Such a cell may further comprise a nucleic acid encoding cas9m and/or a nucleic acid encoding a detectable molecule (e.g., mKate). The interactions of such a circuitry is exemplified in FIG. 8B.

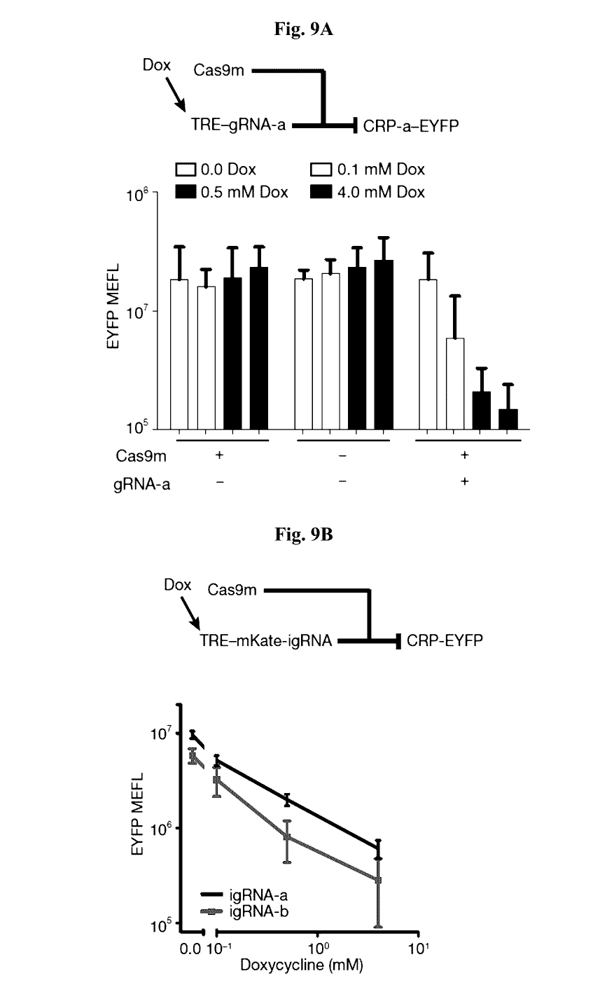

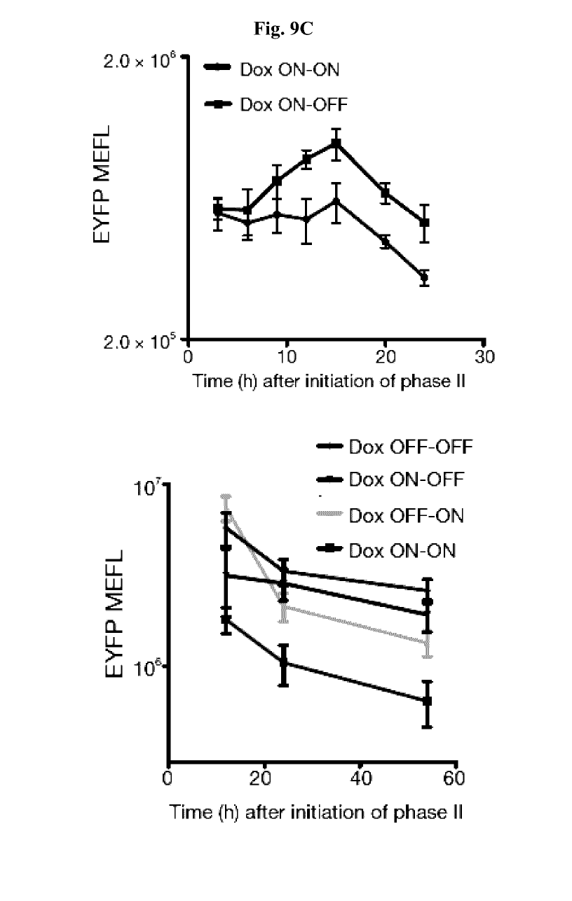

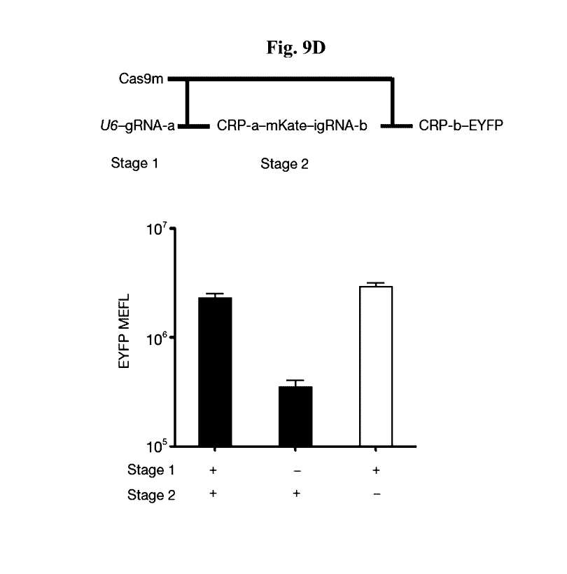

In some embodiments, a cell comprises a nucleic acid comprising an inducible response element (e.g., TRE) as one component of layered circuitry such that the response element allows for reversibility of repression of transcription of nucleic acid encoding a product. For example, doxycycline (Dox) treatment can result in an “ON” mode of the promoter, whereas no Dox results in an “OFF” mode. FIG. 9A-9D exemplify such reversibility of layered circuitry. In some embodiments, the nucleic acid encoding the gRNA is flanked by cognate intronic splice sites, and may be located within a nucleic acid encoding a nucleic acid encoding a product.

A cell may be one of many cells cultured under certain conditions, or part of an organ that is harvested, part of an organoid, or an organism. In some embodiments, a cell disclosed herein is a eukaryotic cell (derived from a eukaryotic organism). In some embodiments, a eukaryotic cell is derived from ectoderm, endoderm, or mesoderm.

In some embodiments, a eukaryotic cell is a human cell. In some embodiments, a eukaryotic cell is a mouse cell, rat cell, cat cell, dog cell, hamster cell, or a cell from a non-human primate.

In some embodiments, a eukaryotic cell derived from ectoderm is derived from surface ectoderm, neural crest or neural tube. In some embodiments, cells of the surface ectoderm are derived from skin (e.g., trichocyte, or keratinocyte) or anterior pituitary (e.g., gonadotrope, corticotrope, thyrotrope, somatotrope, or lactothroph). In some embodiments, cells of the neural crest are derived from peripheral nervous system (e.g., neuro or glial cell), neuroendocrine system (e.g., chromaffin cell, parafollicular cell or glomus cell), skin (melanocyte or Merkel cell), teeth (e.g., odontoblast or cementoblast), or eyes (corneal epithelial cell or photoreceptor cell). In some embodiments, cells of the neural tube are derived from central nervous system (e.g., neuron or astrocyte), ependymal (e.g., ependymocyte), or pineal gland (e.g., pinealocyte).

In some embodiments, a eukaryotic cell derived from endoderm is derived from foregut, pharyngeal pouch (e.g., cells of thyroid gland or paraphyroid gland) or cloaca (e.g., urothelial cell). In some embodiments, cells of the foregut are derived from respiratory system (e.g., pheumocyte, goblet cell or club cell), digestive system, or islets of Langerhans (e.g., alpha cell, beta cell, delta cell or F cell). In some embodiments cells derived from the digestive system are derived from stomach (e.g., G cell, delta cell, enterochromaffin-like cell, gastric chief cell, parietal cell or foveolar cell), intestine (e.g., S cell, delta cell or cholecystokinin cell, goblet cell, paneth cell or tuft cell), liver (e.g., hetaotcyte, hepatic stellate cell or kupffer cell), gall bladder (e.g., cholecystocyte), or exocrine pancreas (e.g., centroacinar cell or pancreatic stellate cell.

In some embodiments, a eukaryotic cell derived from mesoderm is derived from paraxial mesoderm, intermediate mesoderm (e.g., a renal cell or cell of the reproductive system) or lateral plate. In some embodiments, a cell of the paraxial mesoderm a mesenchymal stem cell, such as an osteochondroprogenitor cell (e.g., an osteocyte derived from an osteoblast, a chondrocyte derived from a condroblast), a myofibroblast (e.g., an adipocyte derived from a lipoblast, a myocyte derived from myoblast, tendon cell or cardiac muscle cell) or interstitial cell of Cajal. In some embodiments, a cell of intermediate mesoderm is a renal cell or a cell of reproductive system (e.g., sertoli cell, Leydig cell, granulosa cell, Peg cell or germ cell). A cell of the lateral plate may be a hematopoietic stem cell, or a cell of the circulatory system (e.g., endothelial progenitor cell, endothelial colony forming cell, endotehlial stem cell, angioblast, pericyte or mural cell).

In some embodiments, a cell disclosed herein is a stem cell (e.g., an induced pluripotent stem cell). In some embodiments, a disclosed herein is immortalized (e.g. HEK293, A549, HeLa, Jurkat, 3T3, or Vero cell).

### Libraries

Also provided herein are libraries of composable devices. In some embodiments, provided herein are pairs of engineered nucleic acids, wherein each pair comprises: (a) an engineered nucleic acid comprising a CRISPR-responsive promoter comprising a transcription start site flanked by a first gRNA target site and second gRNA target site, and (b) an engineered nucleic acid comprising a nucleic acid encoding a gRNA, wherein the gRNA binds to the first and second gRNA target sites of the engineered nucleic acid of (a).

In some embodiments, the target sequence of (a) has a length of 15-50 nucleotides (e.g., 20 nucleotides). In some embodiments, the target sequence of (a) has a length of 18-22 nucleotides, or 15-25 nucleotides.

In some embodiments, the gRNA of (b) in the library is operably linked to a promoter. In some embodiments, the promoter to which the gRNA of (b) in the library is operably linked is a constitutive promoter. In some embodiments, the promoter to which the gRNA of (b) in the library is operably linked is a CRP, as provided herein. The promoter to which the gRNA of (b) in the library may be operably linked to any of the promoters described herein.

In some embodiments, the nucleic acid encoding the gRNA of (b) is flanked by cognate intronic splice sites and may be located within a nucleic acid encoding a nucleic acid encoding a product. In some embodiments, the product is a detectable molecule (e.g., a fluorescent protein).

In some embodiments, gRNA and CRP pairs of the library show a repression efficiency from 2-fold to 30-fold (e.g., 2-fold, 4-fold, 15-fold, 26-fold), 1.1-fold to 50-fold (e.g., 1.5-fold, 30-fold, 45-fold, 50-fold), relative to control. In some embodiments, gRNA and CRP pairs of the library show a repression efficiency of at least 30-fold, at least 50-fold, at least a 100-fold, or at least a 1000-fold relative to a control. Repression efficiency herein refers to the ratio of transcription of a nucleic acid encoding a product when transcription is not being repressed (or a control) to the transcription of the nucleic acid encoding a product when repressor devices are used. A control, in some embodiments, is a transcription level of a nucleic acid encoding a product to which a pair of nucleic acids from the library is applied, when either one, or both, of nucleic acids (a) or (b) is absent or rendered non-functional. For example, a control may be a transcription level of a nucleic acid encoding a product observed when gRNA is absent. In some embodiments, a control is a transcription level of a nucleic acid encoding a product when Cas9 (or Cas9m) is absent is a cell. In some embodiments, a control is a transcription level of a nucleic acid encoding a product when the PAM is absent, or the gRNA target sequence is mismatched or scrambled or absent so that the gRNA cannot bind to the gRNA target sites.

In some embodiments, a gRNA target sequence belonging to the library is selected from the following sequences: SEQ ID NO: 29, SEQ ID NO: 30, SEQ ID NO: 31, SEQ ID NO: 32, SEQ ID NO: 33.

In some embodiments, a gRNA target sequence at a hybrid promoter is followed immediately by PAM nucleotide sequence selected from the following sequences: SEQ ID NO: 24, SEQ ID NO: 25, SEQ ID NO: 26, SEQ ID NO: 27, SEQ ID NO: 28 (Table 1).

### EXAMPLES

The following Examples provide data showing that a Cas9 system can be layered to generate circuitry of interconnected transcriptional repression devices. Architectures/constructs for generating a large library of transcriptional devices for such purposes is also provided. The results below demonstrate that CRISPR gRNA can be regulated by RNA Pol II promoters in human cells, which enables incorporation of many Pol II regulatory elements not currently available for Pol III based promoters.

### Example 1

### CRISPR-Responsive Promoters (CRPs) Regulates Expression of Enhanced Yellow Fluorescent Protein (EYFP) in Circuits Wherein gRNA is Constitutively Expressed from a Standard U6 Promoter

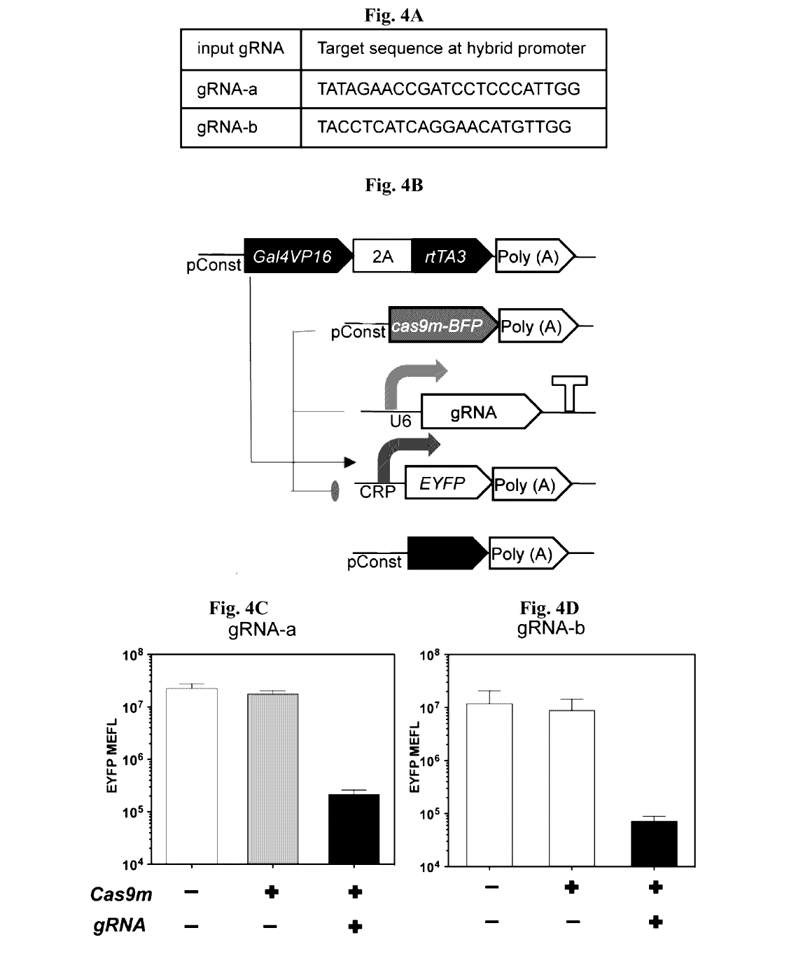

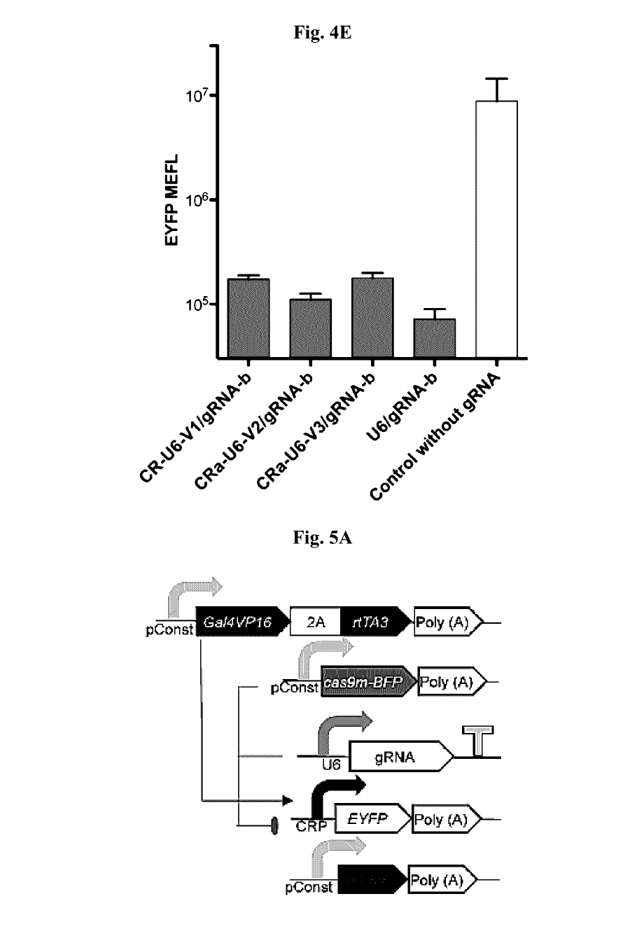

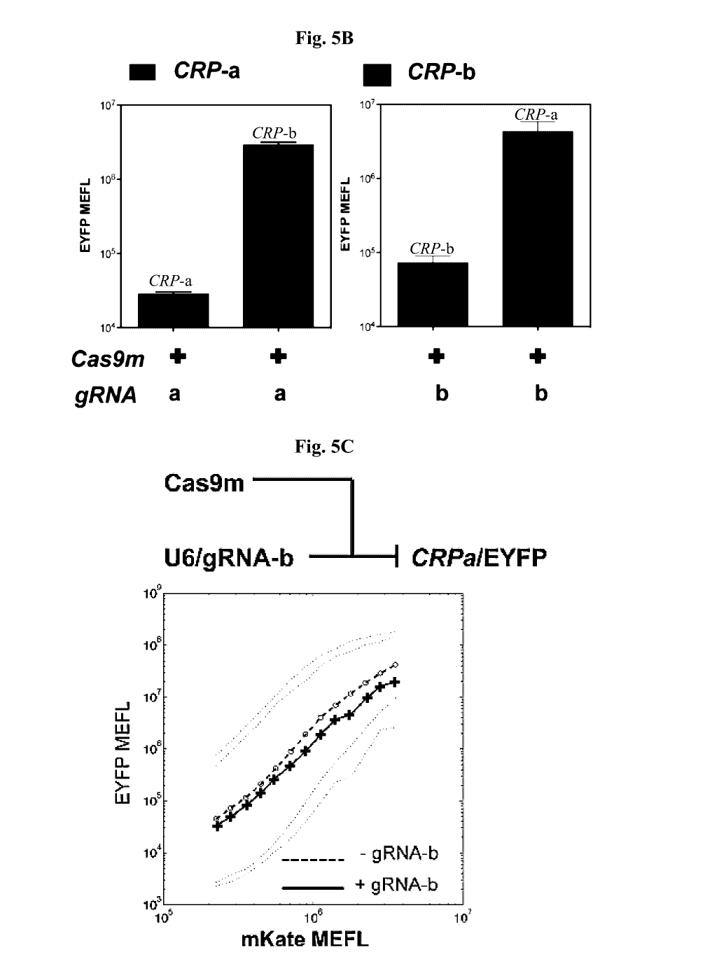

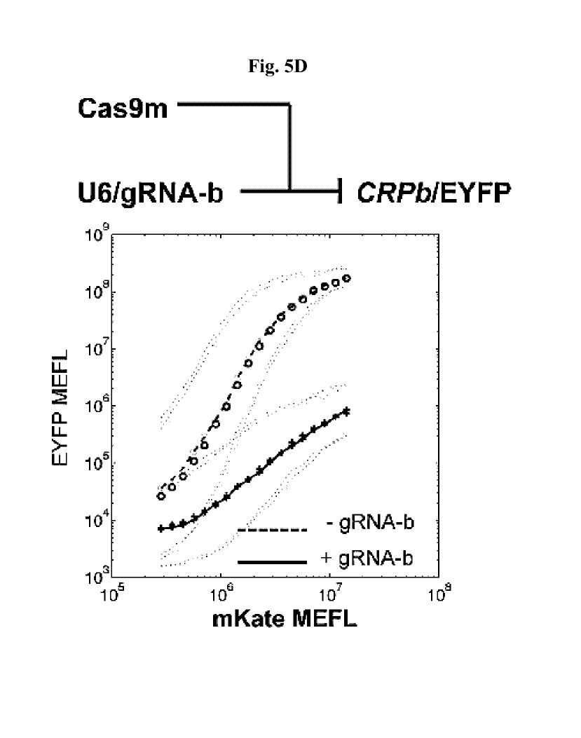

The ability of a CRP to regulate expression of enhanced yellow fluorescent protein (EYFP) based on the presence or absence of gRNA constitutively expressed from a standard U6 promoter was tested. Flow cytometry analysis 48 hours after transfection of regulatory circuitry into human embryonic kidney (HEK) 293 cells showed approximately 100-fold repression for two different gRNA and CRP pairs (gRNA-a and gRNA-b; FIG. 4A-FIG. 4E), and minimal crosstalk between the two devices, demonstrating the desired orthogonality (FIG. 5A-FIG. 5D). The gRNA target site sequences in CRP-a and CRP-b are provided in FIG. 4A. FIG. 4B shows the characterization of a CRP repression device with gRNA expressed from an unmodified U6 promoter. EYFP expression was activated by Ga14VP16 that binds cognate sites at the CRP promoter and was repressed by U6-driven gRNA-mediated targeting of Cas9m to CRPs. mKate fluorescent protein served as the transfection marker. FIGS. 4C-4D show the EYFP output fluorescence for the circuits shown in FIG. 4B for samples transfected with or without Cas9m and U6-driven gRNA-a or gRNA-b. The bar graphs show the mean and standard deviation of EYFP MEFL means for cells expressing >10⁶ MEFL of transfection marker mKate. n=3 biological replicates from one of two representative experiments. FIG. 4E shows a side-by-side comparison of RNA Pol III-driven gRNA-b devices presented throughout this study. The bar graph represents the geometric mean and standard deviation of the EYFP mean in samples transfected with all units of devices (gRNA and Cas9). The light gray bar represents the control group in the absence of any gRNA unit and in the presence of all other units. The CR-U6 devices have comparable efficiency to standard U6. FIG. 5A-FIG. 5D show an analysis of the cross-talk between gRNAs and CRP-a or CRP-b. FIG. 5A illustrates a circuit containing a U6/gRNA repression device together with the gRNA target sites in CRP-a or CRP-b. FIG. 5B presents a comparison of output EYFP MEFL geometric mean when the promoter driving EYFP expression is either CRP-b, which should be targeted by gRNA-b, or CRP-a which should not be targeted by gRNA-b. Bars show the geometric mean and standard deviation of EYFP MEFL means for cells expressing ≧10⁶ MEFL transfection marker mKate for gRNA-b and 3×10⁶ for gRNA-a. n=4 biological replicates pooled from two representative experiments. FIGS. 5C-5D compare details of gRNA-b experiments to a negative control with no gRNA-b, showing output EYFP expression as a function of mKate constitutive fluorescence protein that serves as an indicator of relative circuit copy count. The repression of the matching promoter CRP-b is strong and increases with relative copy count, while the non-matching promoter (CRP-a) shows little effect from inclusion of gRNA-b, thus demonstrating minimal cross-talk from gRNA-b to CRP-a. Values shown are geometric mean (gRNA=black plus, no gRNA=gray circle) and standard deviation (dotted lines) of EYFP expression subpopulations partitioned by constitutive fluorescence. The CRP-b data in FIG. 5B is also reported in FIG. 4D. Herein, one of these two CRPs regulates expression of output reporter EYFP.

### Example 2

### Creation of Layered Circuitry Based on CRISPR Devices

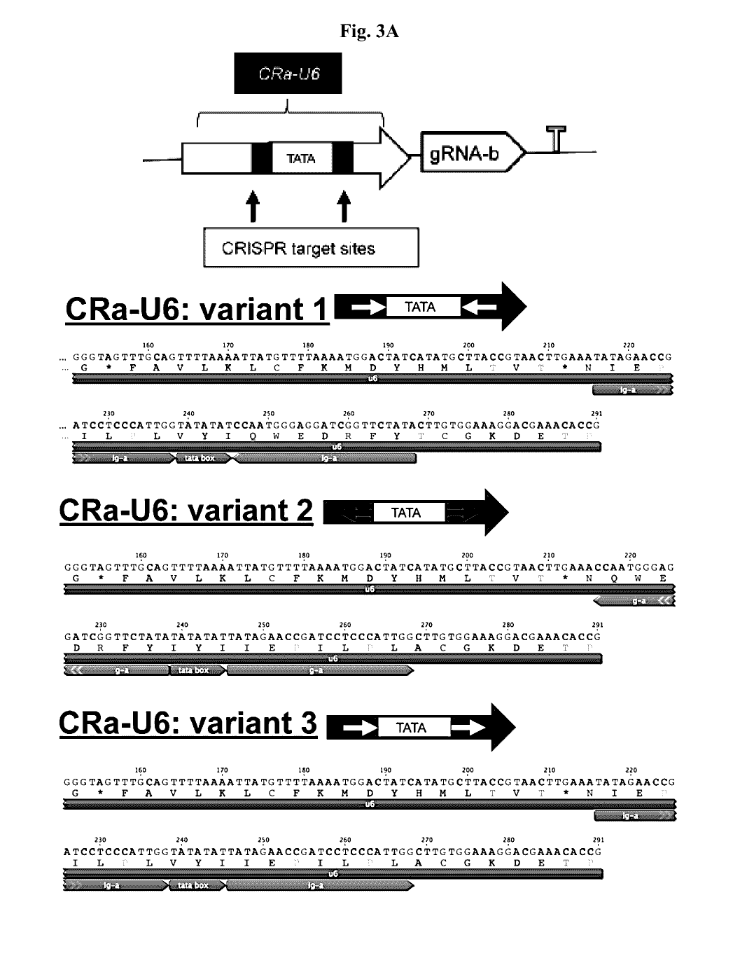

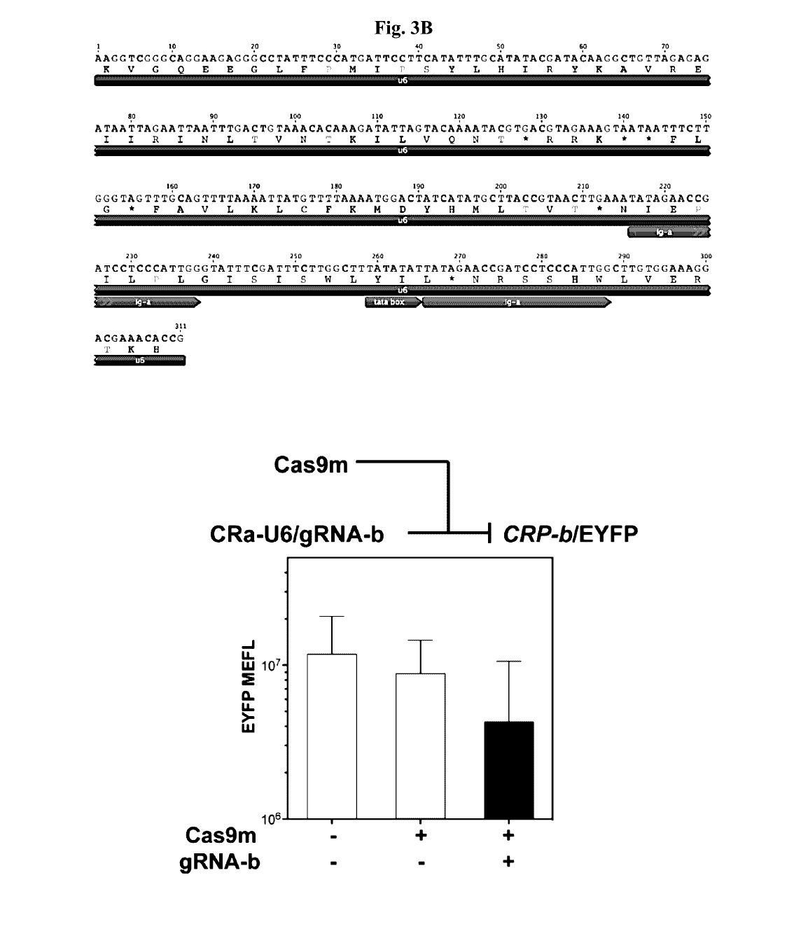

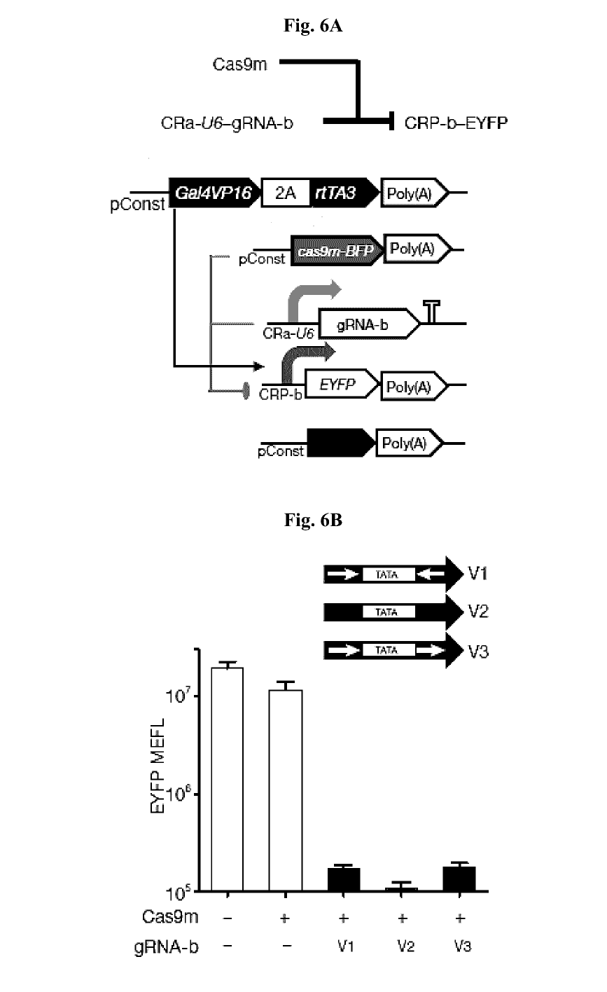

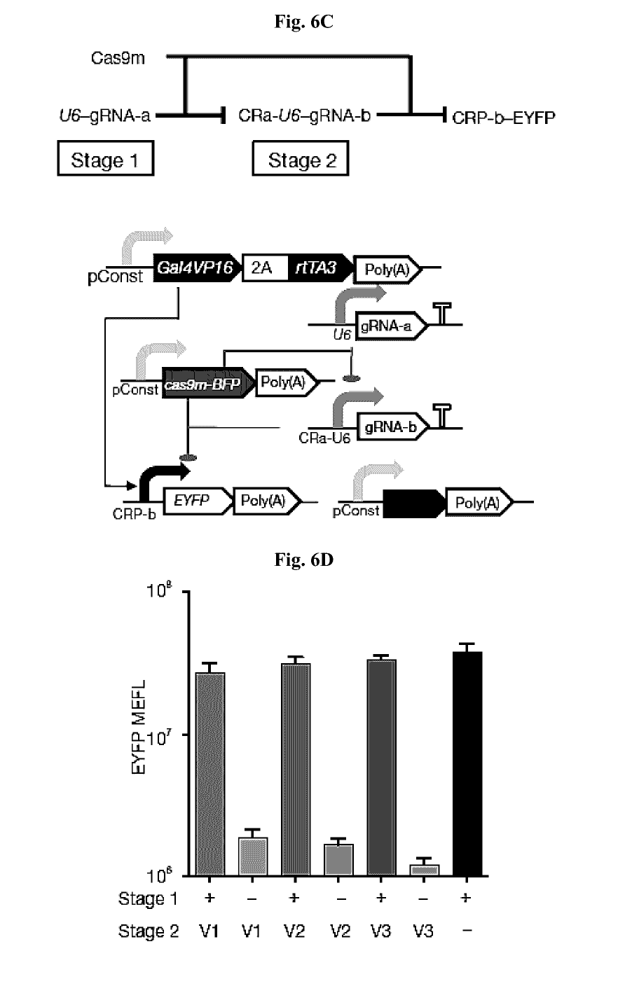

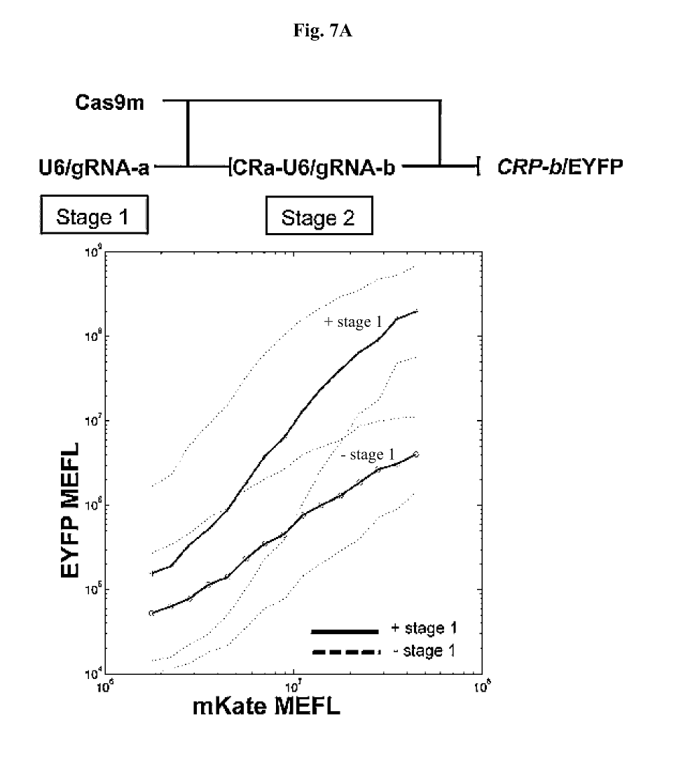

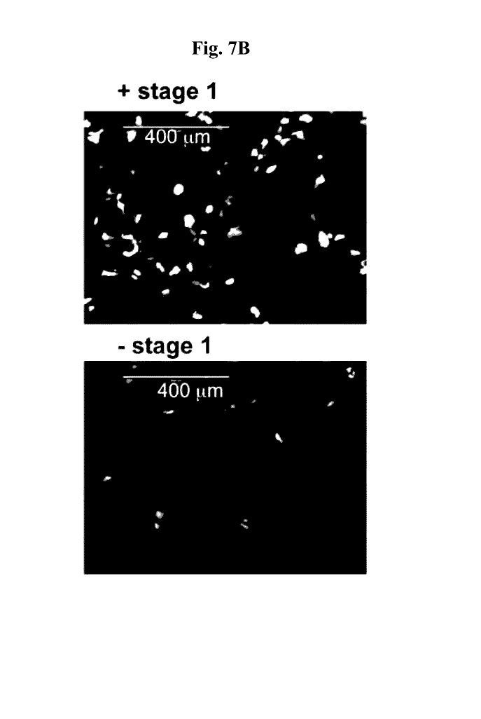

The potential to create layered circuitry based on CRISPR devices was investigated. CR-U6 architecture to both express and be regulated by gRNA was designed to create composable devices. To this end, one Cas9m or gRNA target site was inserted upstream and another was inserted downstream of the U6 promoter TATA box. Three versions of the CRa-U6 promoter differing in the directionality of gRNA-a target sites flanking the TATA box were created (FIG. 3A). Their design is shown in FIG. 3A. Two gRNA-a target sites were inserted within the U6 promoter flanking a TATA sequence. Different variants refer to directionality of gRNA-a target sites flanking the TATA sequence in the U6 promoter. In variant 1, the upstream target site is located at the sense strand (positive) and the downstream site is inserted in the antisense (negative) strand. Variant 2 is negative-positive with respect to the directionality of upstream and downstream target sites. Variant 3 is positive-positive with respect to the directionality of upstream and downstream target sites. Experiments indicate that these regulate the CRP-b promoter with comparable efficiency to the unmodified U6 promoter (FIG. 6A and FIG. 6B). FIG. 6A shows a schematic of a CRP repression device. CRa-U6 drives expression of gRNA-b, which in turn regulates EYEP output. FIG. 6B shows a flow cytometry-based analysis of three repression devices based on CRa-U6-driven gRNA-b expression, in HEK293 cells transfected with the indicated CRa-U6 promoter variants (V1-V3). The geometric mean and standard deviation of means of molecules of equivalent fluorescein (MEFL) of EYFP for cell expressing >3×10⁶ MEFL of transfection marker mKate. n=4 independent technical replicates combined form two experiments. The composability of these three variants in a cascade circuit where U6-driven gRNA-a regulates CRa-U6 expression of gRNA-b, which in turn regulates CRP-b expression of EYFP output (FIG. 6C) was then tested. FIG. 6C is a schematic of CRISPR transcriptional repression device cascade. U6-driven gRNA-a regulates CRa-U6-driven expression gRNA-b, which in turn regulates CRP-b expression of output EYFP. Transfection into HEK293 cells demonstrates highly fiunctional layered CRISPR circuits, exhibiting up to 27 fold de-repression (FIG. 6D and FIG. 7). FIG. 6D shows the EYFP fluorescence for samples transfected either with all transcription units (+for stage 1; V1 or V2 or V3 for stage 2), with all units but without U6-driven gRNA-a (−for stage 1) and with all units but without CRa-U6-driven gRNA-b (+for stage 1; −for stage 2). Data represent geometric mean and standard deviation of means of EYFP MEFL for cells expressing >1×10⁷ MEFL of transfection marker mKate for n=4 biological replicates pooled from two representative experiments. FIG. 7A shows EYFP MEFL as a function of plasmid copy count, as indicated by constitutive fluorescence protein (mKate) MEFL, in the presence or absence of U6/gRNA-a (stage 1) variant 3 as depicted in FIG. 6D. The values on the x-axis (mKate MEFL) are indicative of the relative number of plasmid copies present within each cell. Maximum de-repression of about 50-fold is observed for the highest mKate MEFL. FIG. 7B shows representative microscopy images depicting EYFP output in the presence and absence of stage 1.

### Example 3

### Expression of gRNA from RNA Pol II Promoters

Strategies were developed for expressing gRNA from RNA Pol II promoters, specifically the well-studied mini-CMV promoter, so that CRP devices can be likewise composed. This allows CRISPR devices to be regulated and tuned by commonly used modulators (e.g., Ga14VP16 or rtTA316), and to participate in the same framework as other protein-based regulators, sensors, actuators, and reporters. First, a gRNA sequence was inserted directly downstream of a Tetracycline Response Element (TRE) promoter (FIG. 8A-FIG. 8C). FIG. 8A shows a sequence of RNA Pol II mediated gRNA-a expression. The gRNA sequence downstream of the mini-CMV promoter is in close proximity to transcription initiation site (within 6 nucleotides), followed by a minimal polyadenylation sequence. FIG. 8B depicts a circuit with a repression device using the described promoter. FIG. 8C shows output EYFP, as a function of relative circuit copy count as indicated by constitutive fluorescence protein (mKate) MEFL, showing this repressor device (pluses) induced with Dox as compared to a negative control (circles) where the TRE/gRNA-a plasmid is replaced with an empty plasmid (n=3 biological replicates pooled from three representative experiments). The repressor device shows reduction of EYFP output with addition of Dox, while no effect is observed for the empty plasmid. Values shown are geometric mean and standard deviation (dotted lines) of EYFP expression in subpopulations partitioned by constitutive fluorescence. This design is inspired by RNA Pol II-mediated shRNA expression, which is used for cell context dependent expression and in vivo uses^{16,17}. Flow cytometry analysis 48 hours after transfection of a characterization circuit with gRNA-a transcribed from TRE and controlling CRP-a shows substantial dose-dependent repression upon induction with doxycycline (FIG. 8-FIG. 8C and FIG. 9A). FIG. 9A shows a schematic of a gRNA-a repression device regulated by a TRE promoter and inducible by doxycycline (Dox, top). EYFP output fluorescence was measured for samples transfected with or without Cas9m vector and TRE-driven gRNA-a with the indicated amounts of Dox (bottom). Shown are geometric mean and standard deviation of means of molecules of equivalent fluorescein (MEFL) of EYFP for cells expressing >10⁶ MEFL of transfection marker mKate. n=3 independent technical replicates combined from three experiments.

Reversibility of the circuit was then tested. An increase in EYFP expression was found following removal of doxycycline (FIG. 9C), which confirms reversibility of the circuit. FIG. 9C shows an analysis of reversible repression of the TRE-gRNA-a circuit from FIG. 9A (top) and the TRE/mKate-igRNA-b circuit from FIG. 9B (bottom). Two phases, wherein each phase “ON” indicates induction with 4 mM Dox, and “OFF” indicates zero Dox, were tested. The initial phase was 24 hours (top) or 12 hours (bottom) after transfection. Experimental results are reported for the second phase that includes indicated modulations of Dox. Reversibility is shown by comparison between the different groups. Geometric mean and standard deviation of EYFP MEFL means for cells expressing >10⁶ MEFL of Cas9m-BFP are shown. n=2 independent technical replicates combined from two experiments.

### Example 4

### gRNA as an Intron with Flanking Splicing Sequences

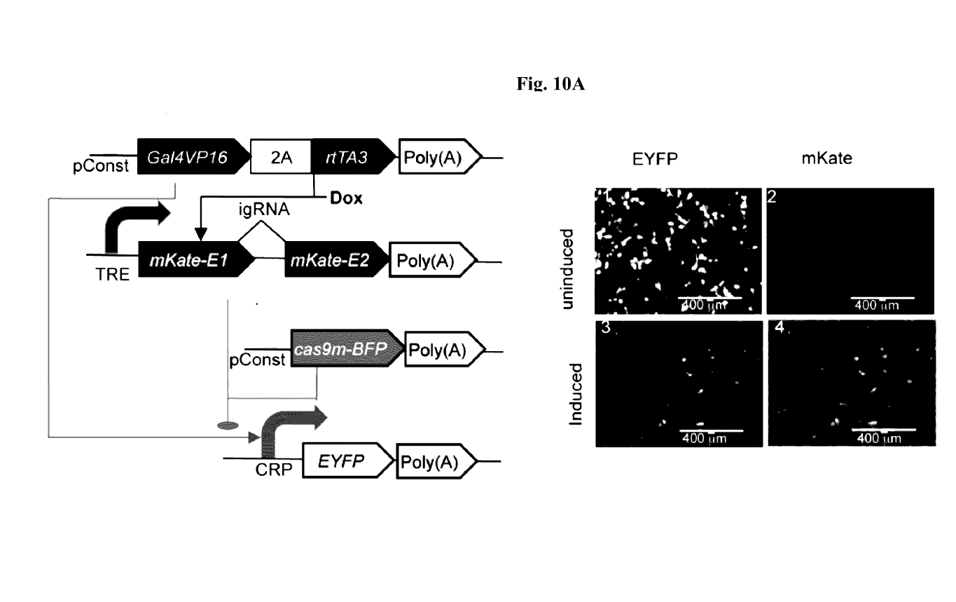

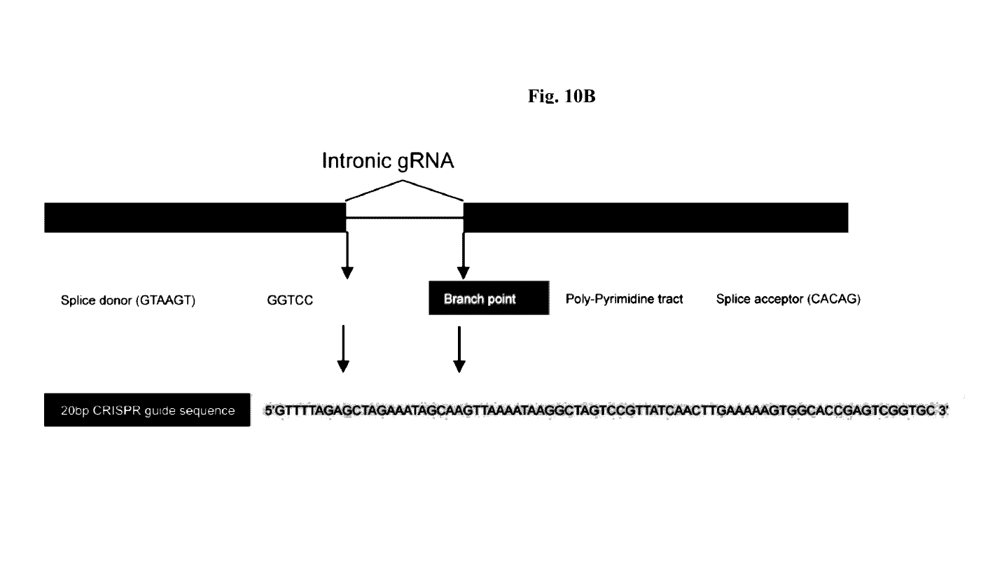

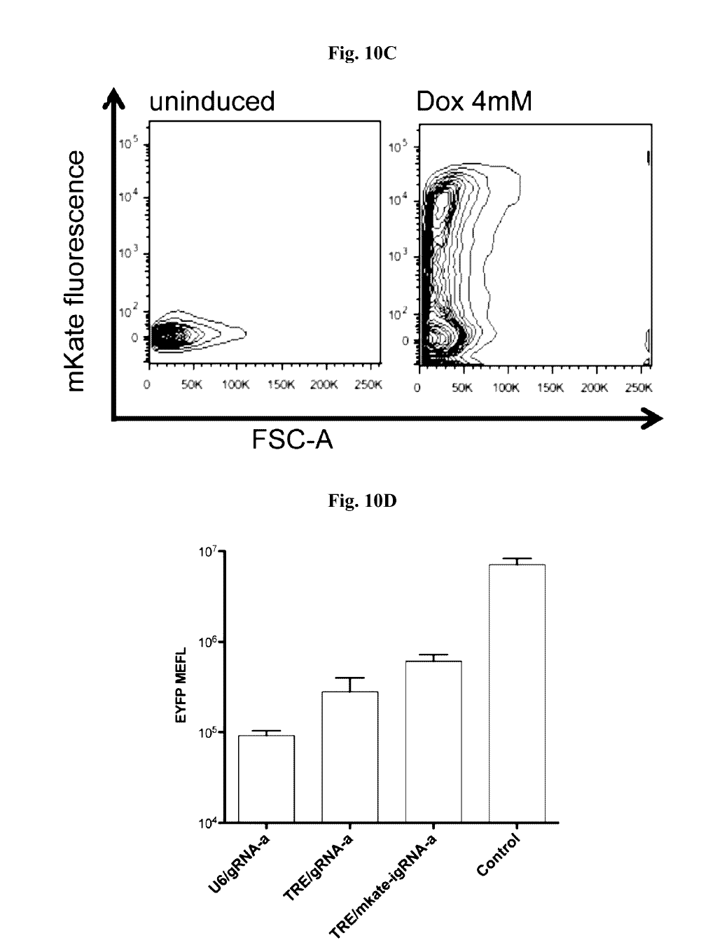

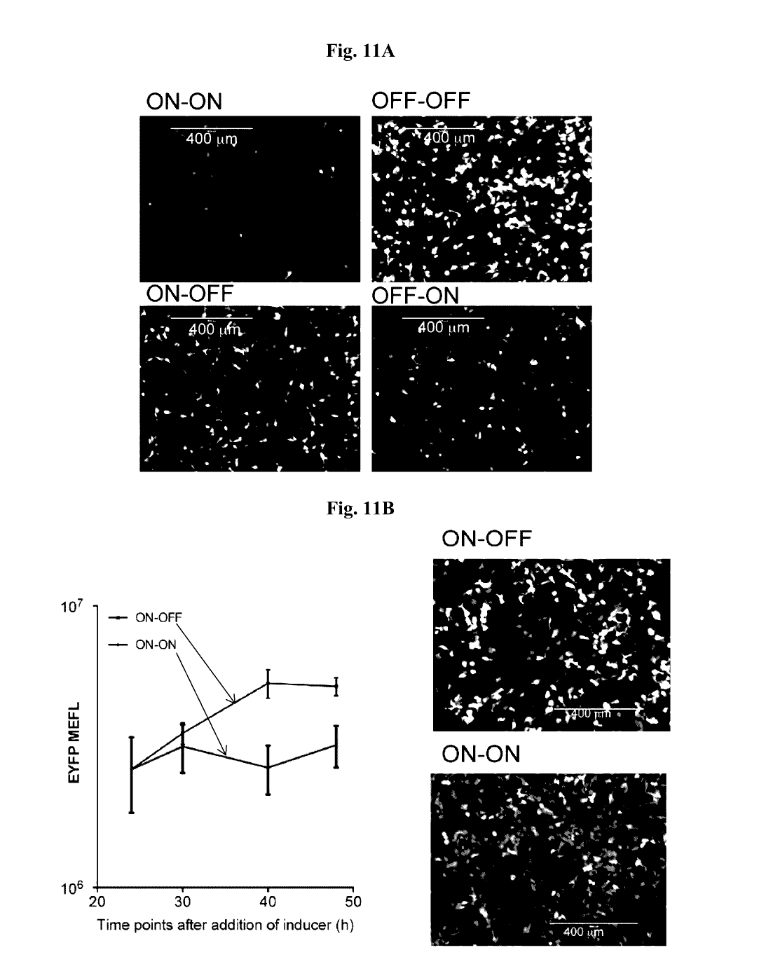

Guide RNA (gRNA) was encoded as an intron with flanking splicing sequences using a strategy similar to that employed for intronic microRNAs (FIG. 10A-FIG. 10D). FIG. 10A shows a circuit with a repression device based on gRNA expressed from an intron inserted within mKate sequence. The four image panels include representative microscopy images for an igRNA-a device in uninduced or fully induced by Dox conditions. Upon addition of Dox, mKate and igRNA-a are expressed (panel 4 versus panel 2) and this represses EYFP (panel 3) in comparison to the uninduced condition (panel 1). FIG. 10B shows a gRNA design as an intron of the mKate fluorescent protein coding gene. An artificial intron is created within a mKate coding sequence using appropriate splicing sequences flanking the gRNA sequence. FIG. 10C shows splicing of the intron via mKate fluorescence as evidenced by flow cytometry after induction of the device with Dox that drives mKate-igRNA expression from the TRE promoter. FIG. 10D shows a side-by-side comparison of RNA Pol 11-driven gRNA-a devices presented throughout this disclosure with standard U6-driven gRNA-a device. The bar graph represents the geometric mean and standard deviation of EYFP MEFL means in samples transfected with all units of devices (gRNA and Cas9) for cells expressing >10⁵ MEFL of the transfection unit, BFP. Control group corresponds to transfection with all transcript units but not gRNAs. Intronic gRNA (igRNA) co-expressed with a protein allows additional capabilities, such as monitoring device regulation by observing a co-expressed fluorescent reporter. Expression of igRNA also potentially allows multiple gRNAs to be expressed from separate introns inserted in a single coding gene. Flow cytometry analysis 48 hours after transfection of characterization circuits for both igRNA-a and igRNA-b show substantial dose-dependent repression upon induction with doxycycline (FIG. 9B), as well as reversibility (FIG. 9C, FIG. 11A and FIG. 11B). FIG. 9B is a schematic of igRNA repression devices expressing mKate and igRNA regulated by the TRE promoter. EYFP fluorescence was measured for samples transfected with circuits containing igRNA-a or igRNA-b devices under the indicated amounts of Dox. Geometric mean and standard deviation of means of EYFP MEFL for cells expressing >10⁶ MEFL of transfection marker mKate are shown. n=2 (gRNA-a) and n=3 (gRNA-b) independent technical replicates combined from two and three experiments, respectively. FIG. 11A shows representative microscopy images of cells transfected with an inducible igRNA-b device as shown in the lower graph of FIG. 9C. Cells were induced with Dox for 12 hours and then divided into four groups according to whether they were continued under Dox induction (ON) or cultured without Dox (OFF). Images were captured 54 hours after the Dox change. FIG. 11B shows an analysis of the reversibility of repression by an igRNA-a device: “ON” samples were induced with 4 mM Dox for 24 hours. The removal of Dox in this condition leads to increased EYFP levels. In the “ON-OFF” group, 24 hours after removal, the level of EYFP is about twice as high as in samples that were maintained in Dox for 48 hours. Data represent geometric mean and standard deviation of EYFP MEFL means for cells expressing >7×10⁷ MEFL of transfection marker BFP. n=3 biological replicates from one representative experiment.

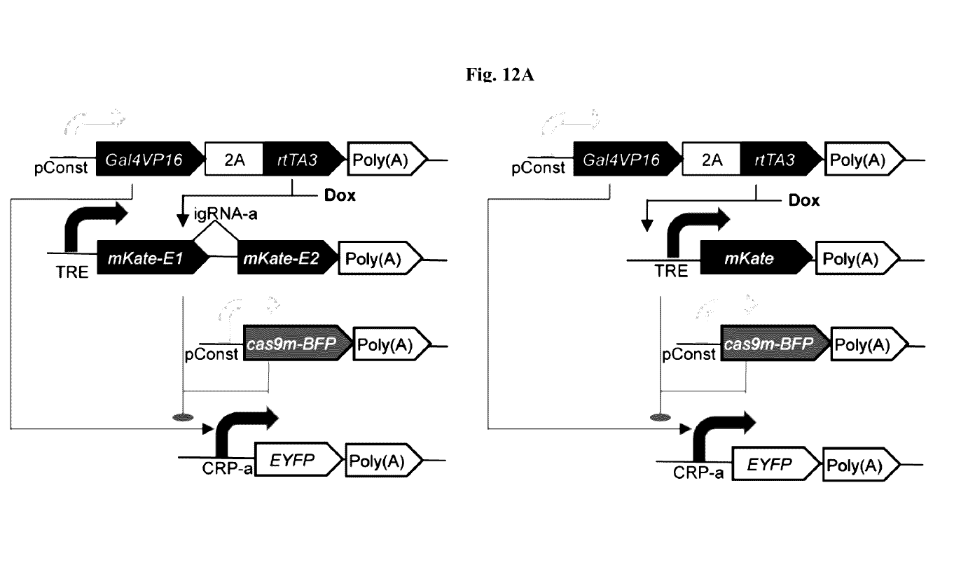

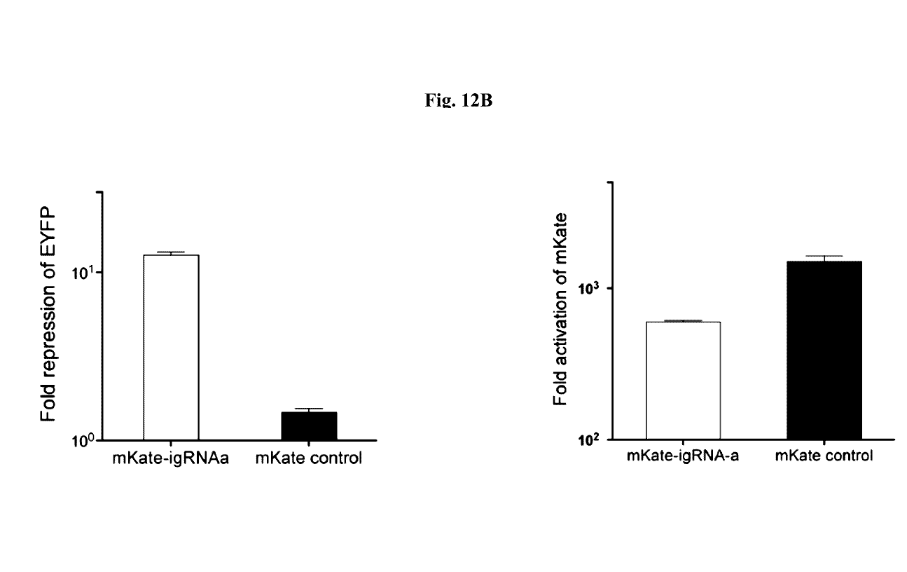

The intronic gRNA (igRNA) devices were assayed further. Two stop codons were inserted within the intron sequence, which allowed verification of proper splicing of intron by observing mKate fluorescence (FIG. 10A-FIG. 10D). To verify that the observed repression of the output is not due to co-expression of the input protein and load on host cellular resources, the igRNA sequence was removed and comparable amount of mKate fluorescent protein was expressed. Significant repression of output in that configuration was not observed (FIG. 12A and FIG. 12B). FIG. 12A shows circuits with two devices, one including igRNA-a expressed as an intron of mKate fluorescence protein (left) and one containing only mKate fluorescence protein expressed under TRE (right). FIG. 12B shows the removal of igRNA-a reduces repression of the devices, suggesting that the observed behavior is not due to load on host cellular resources imposed by accompanying protein expression. Comparing uninduced and fully induced (4 mM Dox) conditions, EYFP expression decreases 12-fold with igRNA-a and only 1.5-fold with TRE/mKate, while mKate expression is induced approximately 1000-fold in both cases. Bars show geometric mean ratio and standard deviation of EYFP MEFL mean ratio for cells expressing ≧10⁵ MEFL constitutive Cas9m-BFP. n=3 biological replicates from one of two representative experiments for TRE/mKate data. The TRE/mKate-igRNA-a data is also reported in FIG. 9B and repeated here for comparison purposes.

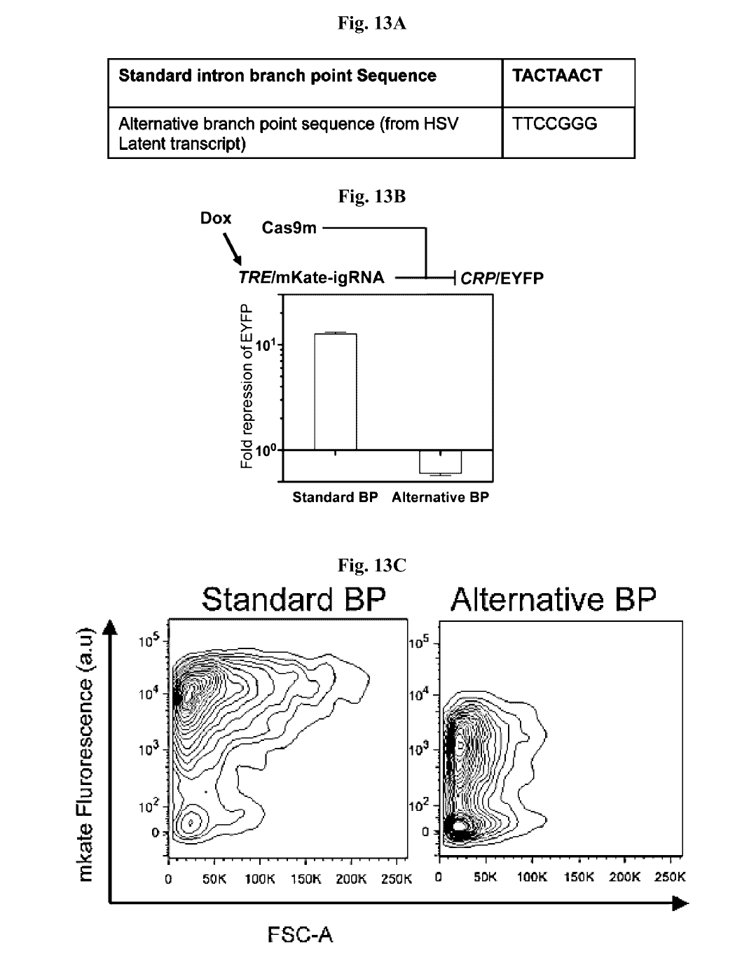

To examine whether proper processing of spliced intron is required for the function of Cas9m/igRNA complex, the eukaryotic intron branch point sequence was replaced with a sequence from HSV latency-associated transcript, which interferes with proper de-branching of a spliced intron. This modification results in diminished igRNA/Cas9m mediated repression efficiency (FIG. 13A-FIG. 13C), suggesting that Cas9m-mediated targeting requires efficient processing of the spliced igRNA. FIG. 13A shows sequences for the widely used intronic branch point that contains the nucleotide A (adenosine) and a branch point sequence from HSV latent transcript that is resistant to de-branching and contains G (Guanosine). FIG. 13B presents a comparison of repression efficiency of devices for igRNA-a with the standard branch point sequence (BP) or igRNA-a with an alternative BP sequence. The bars show geometric mean ratio and standard deviation of EYFP MEFL mean ratio of fully induced (4 mM Dox) and uninduced conditions, for cells expressing ≧10⁵ MEFL of constitutive Cas9m-BFP. n=3 biological replicates pooled from three representative experiments. The standard BP data are also reported in FIG. 9B and FIG. 11 and repeated in FIG. 13B for comparison purposes. FIG. 13C shows mKate fluorescence in a transfected population. The insertion of an alternative branch point sequence decreases mKate expression compared to the standard BP sequence, but the expression levels are comparable.

### Example 5

### Extensibility of CRISPR Regulatory Devices

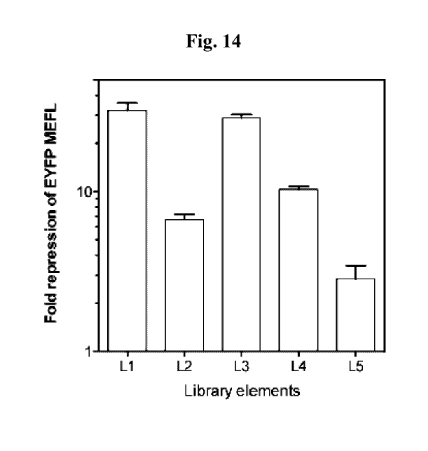

To demonstrate the extensibility of CRISPR regulatory devices, a small library of igRNA and CRP pairs that differ only in the nucleotide sequence of the designated target sites in the CRPs and corresponding intronic gRNA sequences was designed. The nucleotide sequence composition of the target site for each library member is given in Table 1. The library sequences were rationally designed by modification of the gRNA-a target sequence based on current knowledge of CRISPR targeting specificity. Specifically, at least one or more mismatch within the first five nucleotides immediately upstream of PAM that has been shown to be essential for CRISPR specificity was inserted and a number of additional mismatches from nucleotides 5-20 upstream of this sequence were added, with the goal of creating orthogonality within the library. Characterization of this library show a range of repression efficiencies from 2- to 30-fold (FIG. 14), suggesting the feasibility of devices with varying regulatory properties. FIG. 14 shows the fold repression of EYFP (output) in the fully induced samples relative to uninduced conditions. By changing the sequence of the target site, devices with a range of regulatory behaviors can be generated. The bars show the geometric mean ratio and standard deviation of mean ratio of EYFP MEFL in uninduced vs. fully induced samples, for cells expressing ≧10⁷ MEFL transfection marker. n=3 biological replicates pooled from two representative experiments. In the library, no correlation between repression efficiency and AT content of the target sequence nor the N nucleotide identity of NGG (PAM sequence) was detected (data not shown), though such a correlation may become apparent with larger libraries.

TABLE 1
Nucleotide sequences of arget sites of library
members

| | Target sequence at hybrid | |
|---|---|---|
| | promoter followed by PAM | SEQ |
| | nucleotides (bold). | ID |
| ID of gRNA | Note: sequences given 5′ to 3′ | NO: |
| igRNA-L1 | ACGTCAACGTTTCGCACCATCGG | 24 |
| igRNA-L2 | GCTTAATACGGGCTAATCTTGGG | 25 |
| igRNA-L3 | ACTTGGCTACCTCGTTCGACAGG | 26 |
| igRNA-L4 | TTGGCCTACGTACTGCTCTATGG | 27 |
| igRNA-L5 | ACTAGCTATAGATTATCCTAGGG | 28 |

### Example 6

### A Layered CRISPR Cascade with Connected RNA Pol II Promoters with igRNA from a CRP

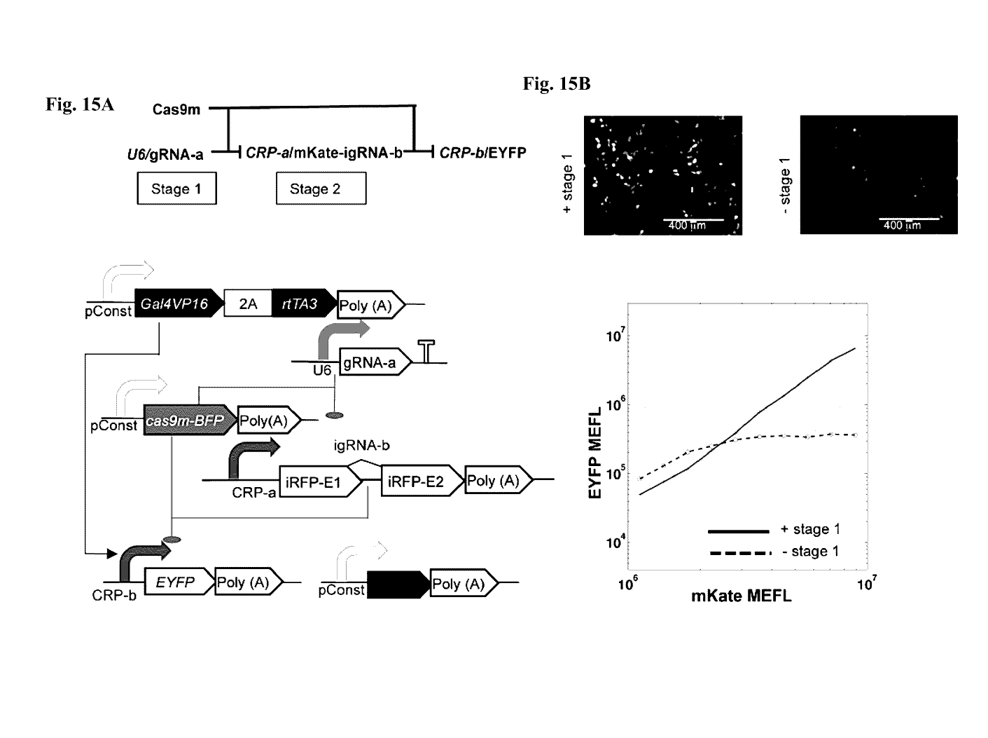

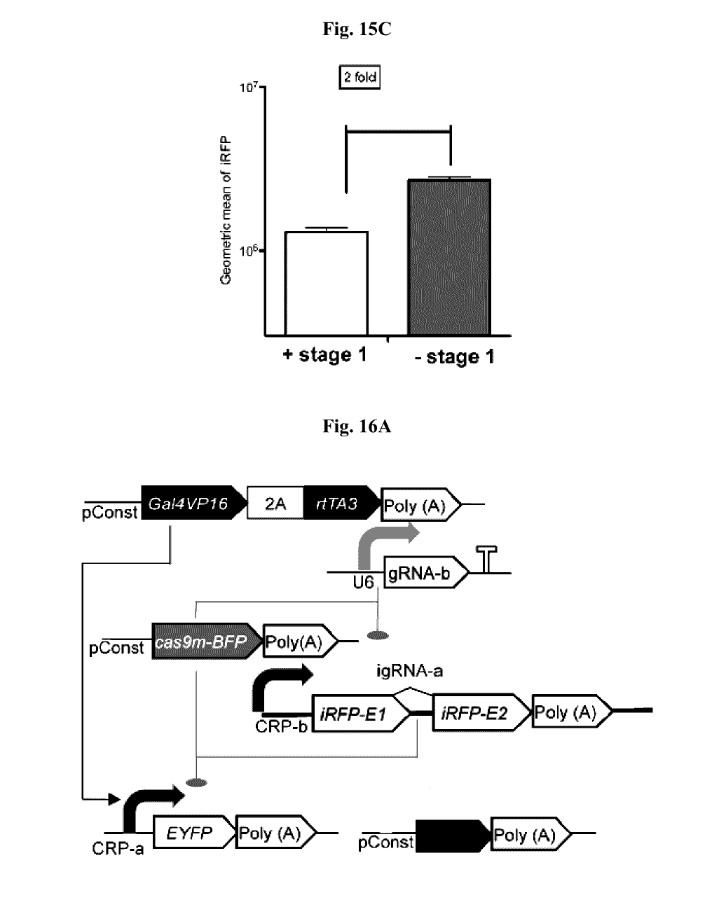

It was next tested whether igRNA from a CRP can regulate another CRP, forming a layered CRISPR cascade with connected RNA Pol-II promoters (FIG. 15A-FIG. 15C). FIG. 15A depicts a cascade of two transcriptional repressors with a U6/gRNA-a device connected to a CRP-a/igRNA-b device. Output EYFP and igRNA-b are both activated by binding of Ga14VP16 to its cognate sites at the CRPs. In the absence of stage 1 (U6-driven gRNA-a), Cas9m is targeted to the CRP-b promoter by igRNA-b expressed from CRP-a as an intron in the iRFP coding sequence. As a result, Cas9m/igRNA-b represses EYFP expression from the CRP-b promoter. In the presence of stage 1, Cas9m- and gRNA-a-mediated targeting to the CRP-a promoter decreases igRNA-b expression, and this alleviates repression of EYFP. FIG. 15B shows output (EYFP MEFL) as a function of relative circuit copy count, as indicated by a constitutive fluorescence protein (mKate MEFL), in the presence or absence of U6/gRNA-a (stage 1). Maximum de-repression of approximately 20-fold is observed at the highest relative circuit copy count. Representative microscopy images illustrate EYFP fluorescence in the presence or absence of stage 1. FIG. 15C shows confirmation of the de-repression effect observed on CRISPR layered circuitry following inclusion of gRNA-a (stage 1) is not due to loss of function of CRISPR after introduction of two gRNAs, through the analysis of. iRFP level (stage 2 protein from which igRNA-b is expressed). Upon addition of stage 1, an approximately 2-fold repression in iRFP expression was observed, which confirms that stage 1 is repressing gRNA-b expression and is in fact layered. Data presented is geometric mean and standard deviation of iRFP mean for cells expressing >3×10⁶ MEFL. n-=4 biological replicates pooled from two representative experiments.

Specifically, the CRa-U6 device in FIG. 6C was replaced by a CRP-a that drives expression of an igRNA-b as an intron of near-infrared fluorescence protein (iRFP) (FIG. 15A-FIG. 15C), which yielded a cascade with repression of about 6-fold (FIG. 9D). FIG. 9D shows a schematic of cascades with igRNA (top). EYFP output fluorescence for samples transfected with or without U6-gRNA-a (stage 1) and CRP-a-igRNA-b (stage 2) (bottom). Geometric mean and standard deviation of EYFP means for cells expressing >3×10⁶ MEFL. n=4 independent technical replicates combined from two experiments.

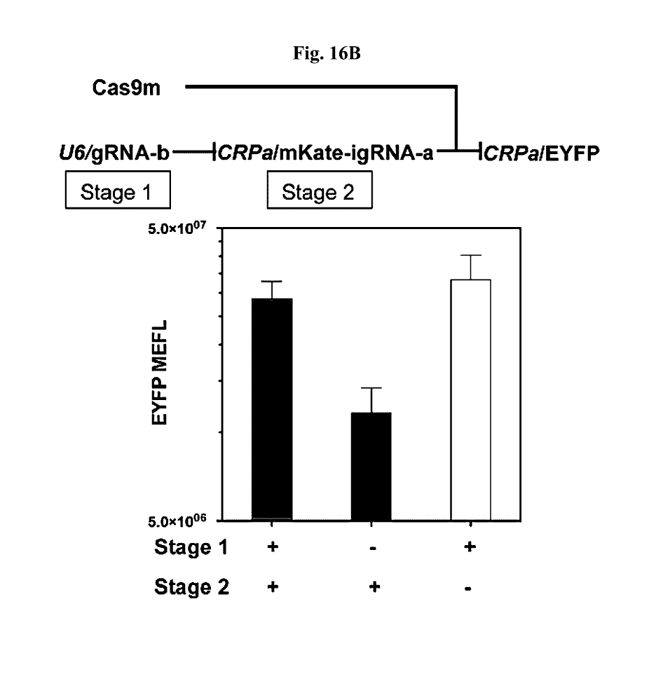

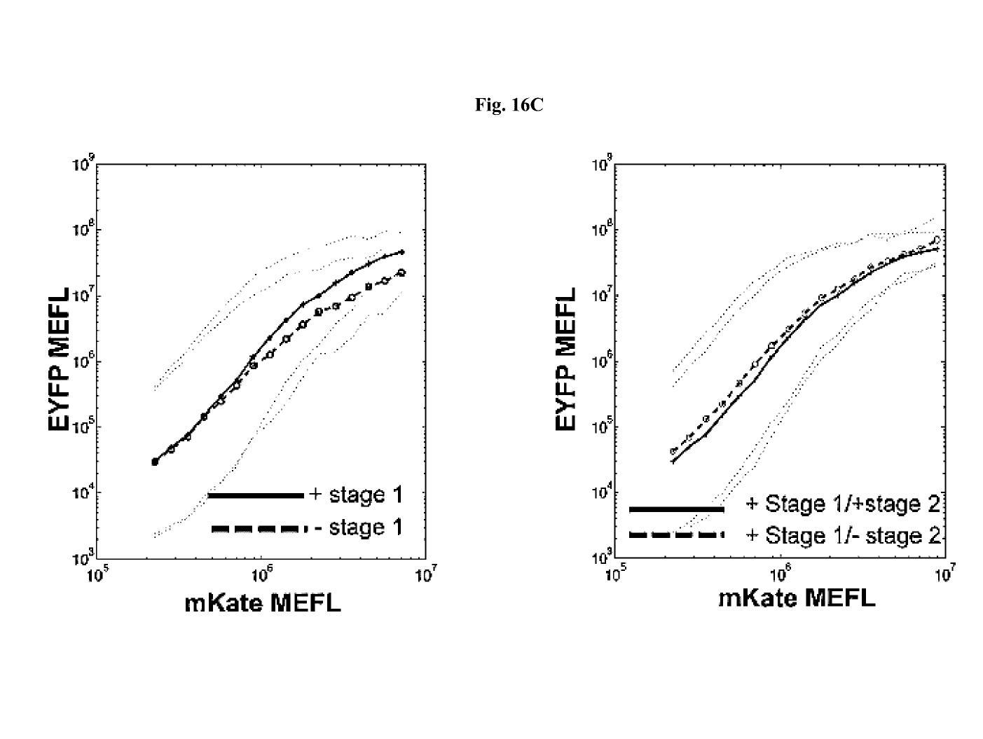

A similar configuration of U6/CRP/CRP promoters, but exchanging gRNA-a and gRNA-b, resulted in a moderately functional cascade (FIG. 16A-FIG. 16C). FIG. 16A shows a cascade of two transcriptional repression devices where U6 promoter-driven gRNA-b represses igRNA-a expression by binding the CRP-b promoter that drives expression of igRNA-a as an intron of iRFP, thus relieving EYFP repression caused by Cas9m and igRNA-a binding its promoter, CRP-a. FIG. 16B shows the output EYFP expression for the above cascade, with approximately a 2.5-fold increase in EYFP level when stage 1 is added. In the absence of igRNA-a (stage 2), EYFP remains high, suggesting minimal direct interference between stage 1 and output CRP-a promoter. The bars show the geometric mean and standard deviation of EYFP MEFL mean for cells expressing ≧10⁶ MEFL transfection marker, mKate. n=3 biological replicates pooled from two representative experiments. FIG. 16C shows the details of EYFP as a function of relative circuit copy count, as indicated by constitutive mKate fluorescence, in the presence and absence of stage 1 (left panel) or in the presence and absence of stage 2 (right panel). The impact of adding stage 1 increases at higher relative circuit copy counts.

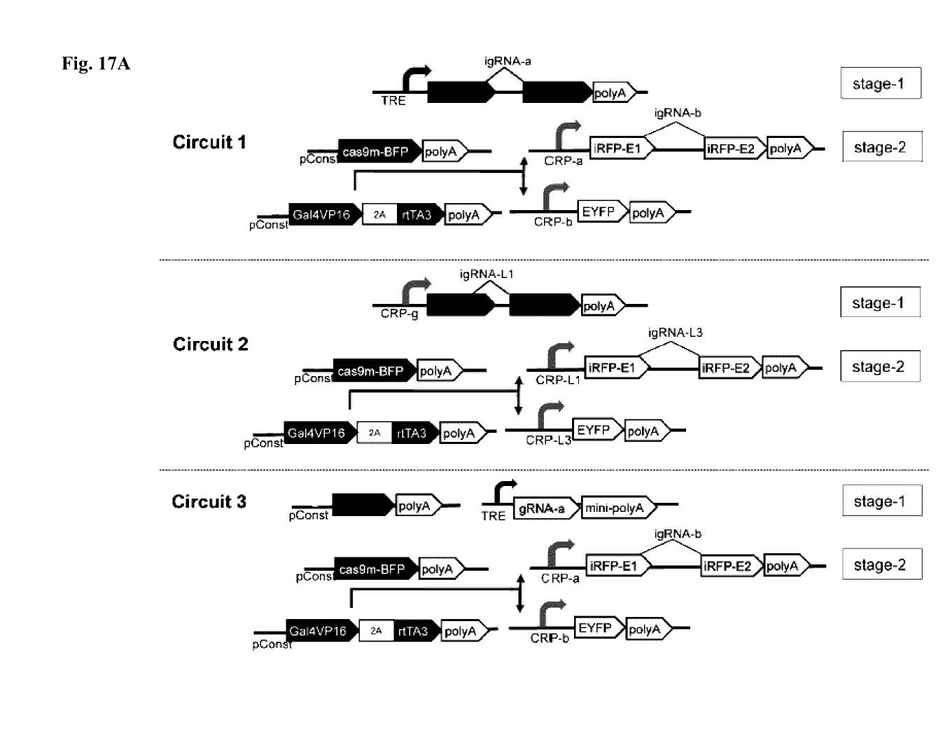

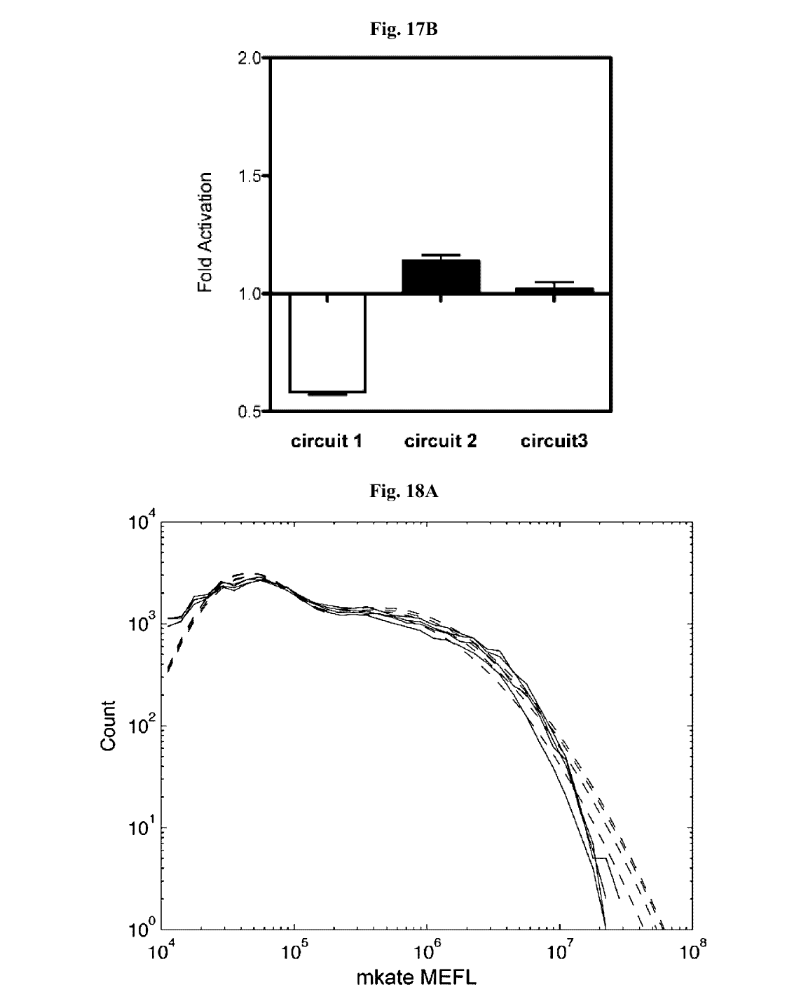

Circuits comprising only RNA Pol II promoters did not yield substantial regulation in this particular experiment (FIG. 17A and FIG. 17B). FIG. 17A shows three different cascades with transcriptional repression devices in which the gRNA of stage 1 is expressed from an RNA Pol II promoter, either as an intron (circuit 1 and 2) or directly (circuit 3). FIG. 17B shows the fold increase in output EYFP for the three cascades was measured. The bars show geometric mean ratio and standard deviation of EYFP MEFL mean ratio of fully induced gRNA-a vs. without igRNA-a, for cells expressing ≧10⁵ MEFL of constitutive Cas9m-BFP. n=3 biological replicates from one of two representative experiments. In this configuration and under the parameters tested, none of the circuits exhibited significant activation.

### Example 7

### Cell Viability Following Transfection with the igRNA-a Device

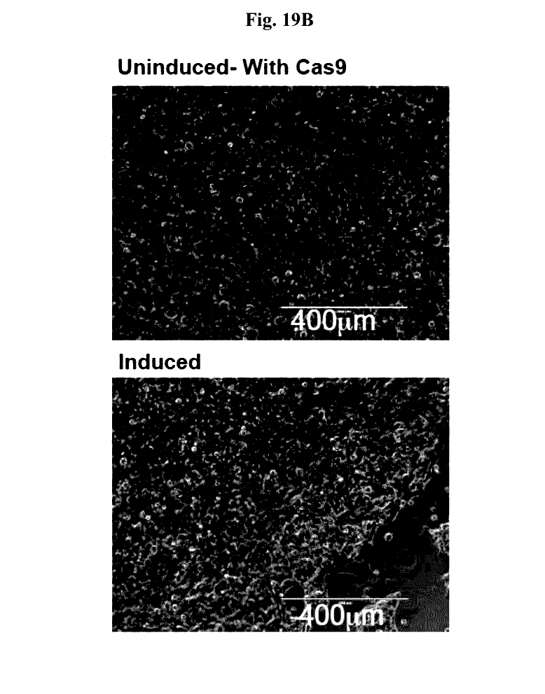

A catalytically inactive mutant Cas9 not fused to any effector domain was used in the studies described herein, otherwise referred to as Cas9m. The absence of an effector domain should reduce deleterious effects in potential off-target binding sites. In addition, the level of transfected Cas9m is kept as low as possible, transfecting 2×10⁵ HEK293 cells with only 70 ng of Cas9m, which has been suggested to decrease off-target effects. Although the efficacy of these strategies is still unclear, they may contribute to the low toxicity observed in the studies described herein. Flow cytometry analysis of cell viability using 7-Aminoactinomycin-D (7-AAD) dye in the igRNA-a device revealed comparable viability across different conditions (FIG. 19A and FIG. 19B). This was also the case for the igRNA-b device. Likewise, microscope observation of transfected cells do not show toxicity beyond levels expected following transfection of the cells (FIG. 19A and FIG. 19B).

### Impact of CRISPR-Based Devices on Host Dynamics

The promoter sequences in the devices reported herein are adapted from a previously reported study evaluating TALEs binding to their cognate target sites and modified to allow CRISPR recognition and targeting. Nucleotide BLAST of these sequences finds no exact matches in the human genome. CRISPR specificity and off-target effects remain active areas of research. The specificity of Cas9 is sequence- and locus-dependent and appears to be governed by the quantity, position and identity of mismatching bases. While prior reports have suggested that 8-12 nucleotides proximal of PAM is essential for specificity, another recent report demonstrates that specificity is defined by exact matching of the PAM-proximal 5 bp to the guide sequence. Studies as the one described herein, and the generation and characterization of larger libraries of engineered regulatory devices will be useful to further improve the understanding of CRISPR specificity.

### Materials and Methods

### Cell Culture and Transfection.

HEK293FT cells were obtained from Invitrogen and maintained in DMEM (CellGro) supplemented with 10% FBS (PAA Laboratories), 1% L-glutamine-streptomycin-penicillin mix (CellGro) and 1% nonesential amino acids (NEAA; HyClone) at 37° C. and 5% CO₂. rtTA3 stable cell lines (HEK293-rtTA3) were created by lentiviral transduction of HEK293 cells with rtTA3 coding sequence under a constitutive promoter and antibiotic selection with hygromycin for 2 weeks. All experiments were done in HEK293-rtTA3 cell lines. Transfections were performed using Attractene reagent (QIAGEN). Cells were seeded the day before at 2×10⁵ cells per well in a 24-well plate. Dosages of plasmids used for the transfections were identified after optimization experiments for each component of the devices and circuits (data not shown). For transfections involving the repression devices, 500 ng of input gRNA plasmid was mixed with a cocktail of other plasmids (ratio of 1x:4x:14x:4x for Ga14VP16-2A-rtTa3 plasmid, EYFP (output) expression plasmid, Cas9m-BFP expression plasmid and mKate expression plasmid, respectively, where x=5 ng) in 70 μl of DMEM (without supplements). For transfection of the cascade circuits of two U6-based devices, 500 ng of the stage 1 gRNA-encoding plasmid was mixed with a cocktail of other plasmids (ratio of 2x:x:14x:10x:5x for Ga14VP16-2A-rtta3 plasmid, EYFP (output) expression plasmid, Cas9m-BFP expression plasmid, stage 2 gRNA plasmid and mKate expression plasmid, respectively, where x=5 ng). For transfections of the cascade circuits of other devices the concentration of the stage 2 gRNA encoding plasmid was twice the value of stage 2 gRNA in U6-only cascades. In control experiments, we replaced the DNA plasmid under study with an equivalent amount of empty DNA plasmid to maintain the total amount of transfected DNA constant among the groups. 1.5 ml of Attractene was added to DNA mixtures, and the tube was gently mixed and kept at room temperature for 20 min to form the DNA-liposome complex. Fresh medium was added to the cells directly before transfection (500 ml of DMEM with supplements). The DNA-Attractene solution was then added drop-wise to the wells. Induction of the circuit was performed at this time as well by addition of doxycycline. In experiments involving the cascade, the ratio of stage 1 gRNA encoding plasmid to the stage 2 gRNA encoding plasmid was 5:1, except for U6-only cascades in which the ratio was 10:1.

Plasmids.

Plasmids used for this project were constructed using the Gateway system (Invitrogen). A plasmid encoding catalytically mutant Cas9 fused to BFP was obtained from Addgene (plasmid 46910). The expression vectors were made by Gateway cloning. The U6-driven gRNA expression cassettes were ordered as gblocks from IDT and cloned into a pCR2.1-TOPO TA vector by Topo TA cloning. The library of CRPs were ordered as gene fragments from IDT and assembled into an appropriate promoter entry vector. igRNA library elements were also ordered as gblocks from IDT and assembled into the mKate entry vector by appropriate restriction digest. Sequences are exemplified below.

Flow Cytometry.

Flow cytometry data were collected 48 h after transfection. ells were trypsinized and centrifuged at 453 g for 5 min t 4° C. The supernatant was then removed, and the cells were resuspended in Hank's Balanced Salt Solution without calcium or magnesium supplemented with 2.5% FBS. BD LSRII was used to obtain low cytometry measurements with the following settings: EBFP, easured with a 405 nm laser and a 450/50 filter; EYFP, measured with a 488 nm laser and a 530/30 filter; mKate, measured with a 61 nm laser and a 695/40 filter. At least 100,000 events were gathered from each sample, ensuring that any 1/10 decade interval with more than 5% of the mean density of events would contain at least 00 expected events.

Statistical Analysis.

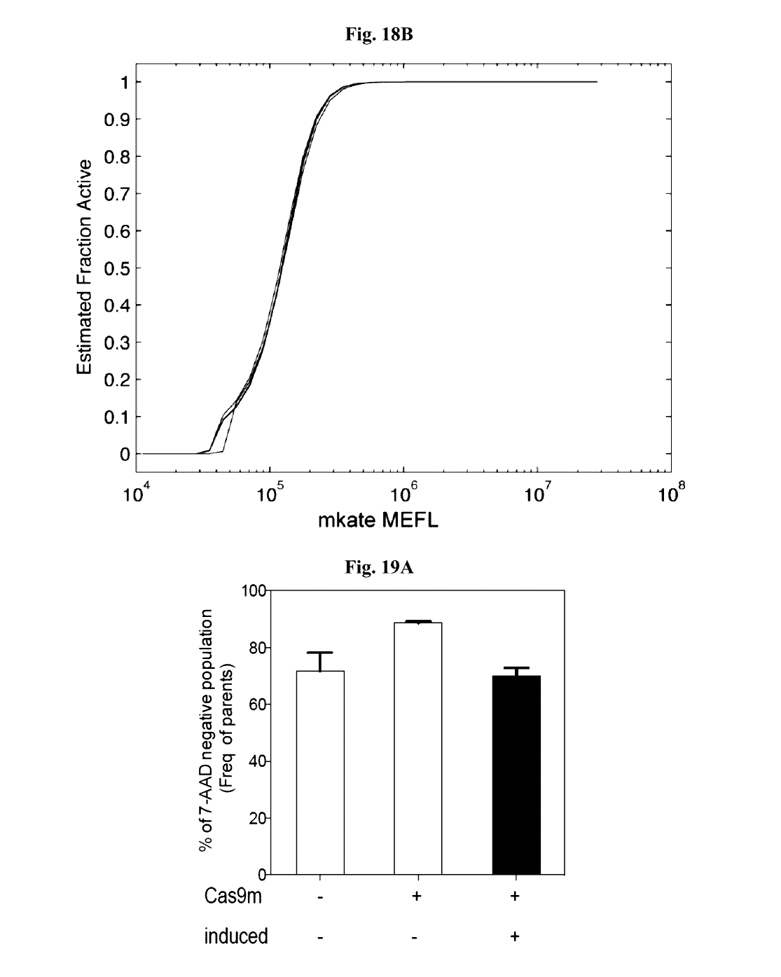

Flow cytometry data were converted from arbitrary units to compensated molecules of equivalent fluorescein (MEFL) using the tool-chain to accelerate synthetic biological engineering (TASBE) characterization method (MIT CSAIL Tech. Report 2012-008 (2012). An affine compensation matrix is computed from single positive and blank controls. FITC measurements are calibrated to MEFL using SpheroTech RCP-30-5-A beads, and mappings from other channels to equivalent FITC are computed from cotransfection of DNA encoding constitutively expressed constitutive EBFP, EYFP and mKate (plus iRFP, for four-color experiments) each controlled by the Hef1a promoter on its own otherwise identical plasmid. Nontransfected controls were included in each experiment. MEFL data are segmented by constitutive fluorescent protein expression into logarithmic bins at 10 bins per decade, and geometric mean and variance are computed for those data points in each bin. Based on the observed constitutive fluorescence distributions (FIG. 18), a threshold was selected as a cutoff for each data set, below which data were excluded as being too close to the non-transfected population. As shown in FIGS. 18A-18B, constitutive fluorescence (mKate) in transient co-transfection typically exhibits a bimodal log normal distribution. FIG. 18A shows the distribution of constitutive fluorescence in a logarithmic histogram with 10 bins/decade for the negative control samples for gRNA-a in FIG. 6B (solid), and a bimodal log normal model fit against each (dashed). FIG. 18B provides an estimate of the fraction of data from successfully transfected cells at any given level of mKate, computed from the bimodal model fit. These are used to set low mKate MEFL cutoffs, below which data is discarded.

Data shown in the figures are geometric mean and s.d. of means for cells expressing the transfection marker mKate based on the MEFL threshold set. High outliers were removed by excluding all bins without at least 100 data points. Both population and per-bin geometric statistics were computed over this filtered set of data. Sample sizes were predetermined for each experiment based on initial pilot experiments. We also ensured that we gathered at least 100,000 flow cytometry events per technical replicate. During analysis of flow cytometry data, samples were excluded by the following predetermined criteria: if they contained less than 10% of the number of events or less than 10% of the fraction of successful transfections of the mode for the batch in which they were collected.

SEQUENCES

U6-gRNA-a
(SEQ ID NO: 1)
AAGGTCGGGCAGGAAGAGGGCCTATTTCCCATGATTCCTTCATATTTGCA
TATACGATACAAGGCTGTTAGAGAGATAATTAGAATTAATTTGACTGTAA
ACACAAAGATATTAGTACAAAATACGTGACGTAGAAAGTAATAATTTCTT
GGGTAGTTTGCAGTTTTAAAATTATGTTTTAAAATGGACTATCATATGCT
TACCGTAACTTGAAAGTATTTCGATTTCTTGGCTTTATATATCTTGTGGA
AAGGACGAAACACCGTATAGAACCGATCCTCCCATGTTTTAGAGCTAGAA
ATAGCAAGTTAAAATAAGGCTAGTCCGTTATCAACTTGAAAAAGTGGCAC
CGAGTCGGTGCTTTTTTT
U6-gRNA-b
(SEQ ID NO: 2)
AAGGTCGGGCAGGAAGAGGGCCTATTTCCCATGATTCCTTCATATTTGCA
TATACGATACAAGGCTGTTAGAGAGATAATTAGAATTAATTTGACTGTAA
ACACAAAGATATTAGTACAAAATACGTGACGTAGAAAGTAATAATTTCTT
GGGTAGTTTGCAGTTTTAAAATTATGTTTTAAAATGGACTATCATATGCT
TACCGTAACTTGAAAGTATTTCGATTTCTTGGCTTTATATATCTTGTGGA
AAGGACGAAACACCGTACCTCATCAGGAACATGTGTTTAAGAGCTATGCT
GGAAACAGCAGAAATAGCAAGTTTAAATAAGGCTAGTCCGTTATCAACTT
GAAAAAGTGGCACCGAGTCGGTGCTTTTTTT
CRa-U6/gRNA-b (Version 1)
(SEQ ID NO: 3)
GGTTTACCGAGCTCTTATTGGTTTTCAAACTTCATTGACTGTGCCAAGGT
CGGGCAGGAAGAGGGCCTATTTCCCATGATTCCTTCATATTTGCATATAC
GATACAAGGCTGTTAGAGAGATAATTAGAATTAATTTGACTGTAAACAAA
GATATTAGTACAAAATACGTGACGTAGAAAGTAATAATTTCTTGGGTAGT
TTGCAGTTTTAAAATTATGTTTTAAAATGGACTATCATATGCTTACCGTA
ACTTGAAATATAGAACCGATCCTCCCATTGGTATATATCCAATGGGAGGA
TCGGTTCTATACTTGTGGAAAGGACGAAACACCGTACCTCATCAGGAACA
TGTGTTTAAGAGCTATGCTGGAAACAGCAGAAATAGCAAGTTTAAATAAG
GCTAGTCCGTTATCAACTTGAAAAAGTGGCACCGAGTCGGTGCTTTTTTT
GGTGCGTTTTTATGCTTGTAGTATTGTATAATGTTTTT
CRa-U6/gRNA-b (version 2)
(SEQ ID NO: 4)
AAGGTCGGGCAGGAAGAGGGCCTATTTCCCATGATTCCTTCATATTTGCA
TATACGATACAAGGCTGTTAGAGAGATAATTAGAATTAATTTGACTGTAA
ACACAAAGATATTAGTACAAAATACGTGACGTAGAAAGTAATAATTTCTT
GGGTAGTTTGCAGTTTTAAAATTATGTTTTAAAATGGACTATCATGCTTA
CCGTAACTTGAAACCAATGGGAGGATCGGTTCTATATATATATTATAGAA
CCGATCCTCCCATTGGCTTGTGGAAAGGACGAAACACCGTACCTCATCAG
GAACATGTGTTTAAGAGCTATGCTGGAAACAGCAGAAATAGCAAGTTTAA
ATAAGGCTAGTCCGTTATCAACTTGAAAAAGTGGCACCGAGTCGGTGCTT
TTTTT
CRa-U6/gRNA-b (version 3)
(SEQ ID NO: 5)
GGTTTACCGAGCTCTTATTGGTTTTCAAACTTCATTGACTGTGCCAAGGT
CGGGCAGGAAGAGGGCCTATTTCCCATGATTCCTTCATATTTGCATATAC
GATACAAGGCTGTTAGAGAGATAATTAGAATTAATTTGACTGTAAACACA
AAGATATTAGTACAAAATACGTGACGTAGAAAGTAATAATTTCTTGGGTA
GTTTGCAGTTTTAAAATTATGTTTTAAAATGGACTATCATATGCTTACCG
TAACTTGAAATATAGAACCGATCCTCCCATTGGTATATTATAGAACCGAT
CCTCCCATTGGCTTGTGGAAAGGACGAAACACCGTACCTCATCAGGAACA
TGTGTTTAAGAGCTATGCTGGAAACAGCAGAAATAGCAAGTTTAAATAAG
GCTAGTCCGTTATCAACTTGAAAAAGTGGCACCGAGTCGGTGCTTTTTTT
GGTGCGTTTTTATGCTTGTAGTATTGTATAATGTTTTT
mKate-Intronic gRNA-b
(SEQ ID NO: 6)
TCTAAGGGCGAAGAGCTGATTAAGGAGAACATGCACATGAAGCTGTACAT
GGAGGGCACCGTGAACAACCACCACTTCAAGTGCACATCCGAGGGCGAAG
GCAAGCCCTACGAGGGCACCCAGACCATGAGAATCAAGGTGGTCGAGGGC
GGCCCTCTCCCCTTCGCCTTCGACATCCTGGCTACCAGCTTCATGTACGG
CAGCAAAACCTTCATCAACCACACCCAGGGCATCCCCGACTTCTTTAAGC
AGTCCTTCCCTGAGGTAAGTGGTCCTACCTCATCAGGAACATGTGTTTTA
GAGCTAGAAATAGCAAGTTAAAATAAGGCTAGTCCGTTATCAACTTGAAA
AAGTGGCACCGAGTCGGTGCTACTAACTCTCGAGTCTTCTTTTTTTTTTT
CACAGGGCTTCACATGGGAGAGAGTCACCACATACGAAGACGGGGGCGTG
CTGACCGCTACCCAGGACACCAGCCTCCAGGACGGCTGCCTCATCTACAA
CGTCAAGATCAGAGGGGTGAACTTCCCATCCAACGGCCCTGTGATGCAGA
AGAAAACACTCGGCTGGGAGGCCTCCACCGAGATGCTGTACCCCGCTGAC
GGCGGCCTGGAAGGCAGAAGCGACATGGCCCTGAAGCTCGTGGGCGGGGG
CCACCTGATCTGCAACTTGAAGACCACATACAGATCCAAGAAACCCGCTA
AGAACCTCAAGATGCCCGGCGTCTACTATGTGGACAGAAGACTGGAAAGA
ATCAAGGAGGCCGACAAAGAGACCTACGTCGAGCAGCACGAGGTGGCTGT
GGCCAGATACTGCG
mKate intronic gRNA-a
(SEQ ID NO: 7)
ATGGTGTCTAAGGGCGAAGAGCTGATTAAGGAGAACATGCACATGAAGCT
GTACATGGAGGGCACCGTGAACAACCACCACTTCAAGTGCACATCCGAGG
GCGAAGGCAAGCCCTACGAGGGCACCCAGACCATGAGAATCAAGGTGGTC
GAGGGCGGCCCTCTCCCCTTCGCCTTCGACATCCTGGCTACCAGCTTCAT
GTACGGCAGCAAAACCTTCATCAACCACACCCAGGGCATCCCCGACTTCT
TTAAGCAGTCCTTCCCTGAGGTAAGTGGTCCTATAGAACCGATCCTCCCA
TGTTTTAGAGCTAGAAATAGCAAGTTAAAATAAGGCTAGTCCGTTATCAA
CTTGAAAAAGTGGCACCGAGTCGGTGCTACTAACTCGAGTCTTCTTTTTT
TTTTTCACAGGGCTTCACATGGGAGAGAGTCACCACATACGAAGACGGGG
GCGTGCTGACCGCTACCCAGGACACCAGCCTCCAGGACGGCTGCCTCATC
TACAACGTCAAGATCAGAGGGGTGAACTTCCCATCCAACGGCCCTGTGAT
GCAGAAGAAAACACTCGGCTGGGAGGCCTCCACCGAGATGCTGTACCCCG
CTGACGGCGGCCTGGAAGGCAGAAGCGACATGGCCCTGAAGCTCGTGGGC
GGGGGCCACCTGATCTGCAACTTGAAGACCACATACAGATCCAAGAAACC
CGCTAAGAACCTCAAGATGCCCGGCGTCTACTATGTGGACAGAAGACTGG
AAAGAATCAAGGAGGCCGACAAAGAGACCTACGTCGAGCAGCACGAGGTG
GCTGTGGCCAGATACTGCGACCTCCCTAGCAAACTGGGGCACAAACTTAA
TTGA
CRpA promoter
(SEQ. ID NO: 8)
GCTCCGAATTTCTCGACAGATCTCATGTGATTACGCCAAGCTACGGGCGG
AGTACTGTCCTCCGAGCGGAGTACTGTCCTCCGAGCGGAGTACTGTCCTC
CGAGCGGAGTACTGTCCTCCGAGCGGAGTTCTGTCCTCCGAGCGGAGACT
CTAGATATAGAACCGATCCTCCCATTGGAATTCTAGGCGTGTACGGTGGG
AGGCCTATATAAGCAGAGCTCGTTTAGTGAACCGTCAGATCGCCTCGAGT
ATAGAACCGATCCTCCCATTGGATCCAATTCGAC
CRP-b promoter
(SEQ ID NO: 9)
GCTCCGAATTTCTCGACAGATCTCATGTGATTACGCCAAGCTACGGGCGG
AGTACTGTCCTCCGAGCGGAGTACTGTCCTCCGAGCGGAGTACTGTCCTC
CGAGCGGAGTACTGTCCTCCGAGCGGAGTTCTGTCCTCCGAGCGGAGACT
CTAGATACCTCATCAGGAACATGTTGGAATTCTAGGCGTGTACGGTGGGA
GGCCTATATAAGCAGAGCTCGTTTAGTGAACCGTCAGATCGCCTCGAGTA
CCTCATCAGGAACATGTTGGATCCAATTCGACC
iRFP-igRNA-a
(SEQ ID NO: 10)
ATGGCTGAAGGATCCGTCGCCAGGCAGCCTGACCTCTTGACCTGCGACGA
TGAGCCGATCCATATCCCCGGTGCCATCCAACCGCATGGACTGCTGCTCG
CCCTCGCCGCCGACATGACGATCGTTGCCGGCAGCGACAACCTTCCCGAA
CTCACCGGACTGGCGATCGGCGCCCTGATCGGCCGCTCTGCGGCCGATGT
CTTCGACTCGGAGACGCACAACCGTCTGACGATCGCCTTGGCCGAGCCCG
GGGCGGCCGTCGGAGCACCGATCACTGTCGGCTTCACGATGCGAAAGGTA
AGTGGTCCTATAGAACCGATCCTCCCATGTTTTAGAGCTAGAAATAGCAA
GTTAAAATAAGGCTAGTCCGTTATCAACTTGAAAAAGTGGCACCGAGTCG
GTGCTACTAACTCTCGAGTCTTCTTTTTTTTTTTCACAGGACGCAGGCTT
CATCGGCTCCTGGCATCGCCATGATCAGCTCATCTTCCTCGAGCTCGAGC
CTCCCCAGCGGGACGTCGCCGAGCCGCAGGCGTTCTTCCGCCGCACCAAC
AGCGCCATCCGCCGCCTGCAGGCCGCCGAAACCTTGGAAAGCGCCACGCC
GCCGCGGCGCAAGAGGTGCGGAAGATTACCGGCTTCGATCGGGTGATGAT
CTATCGCTTCGCCTCCGACTTCAGCGGCGAAGTGATCGCAGAGGATCGGT
GCGCCGAGGTCGAGTCAAAACTAGGCCTGCACTATCCTGCCTCAACCGTG
CCGGCGCAGGCCCGTCGGCTCTATACCATCAACCCGGTACGGATCATTCC
CGATATCAATTATCGGCCGGTGCCGGTCACCCCAGACCTCAATCCGGTCA
CCGGGCGGCCGATTGATCTTAGCTTCGCCATCCTGCGCAGCGTCTCGCCC
GTCCATCTGGAATTCATGCGCAACATAGGCATGCACGGCACGATGTCGAT
CTCGATTTTTCGCGGCGAGCGACTGTGGGGATTGATCGTTTGCCATCACC
GAACGCCGTACTACGTCGATCTCGATGGCCGCCAAGCCTGCGAGCTAGTC
GCCCAGGTTCTGGCCTGGCAGATCGGCGTGATGGAAGAGTGA
iRFP-igRNA-b
(SEQ ID NO: 11)
ATGGCTGAAGGATCCGTCGCCAGGCAGCCTGACCTCTTGACCTGCGACGA
TGAGCCGATCCATATCCCCGGTGCCATCCAACCGCATGGACTGCTGCTCG
CCCTCGCCGCCGACATGACGATCGTTGCCGGCAGCGACAACCTTCCCGAA
CTCACCGGACTGGCGATCGGCGCCCTGATCGGCCGCTCTGCGGCCGATGT
CTTCGACTCGGAGACGCACAACCGTCTGACGATCGCCTTGGCCGAGCCCG
GGGCGGCCGTCGGAGCACCGATCACTGTCGGCTTCACGATGCGAAAGGTA
AGTGGTCCTACCTCATCAGGAACATGTGTTTTAGAGCTAGAAATAGCAAG
TTAAAATAAGGCTAGTCCGTTATCAACTTGAAAAAGTGGCACCGAGTCGG
TGCTACTAACTCTCGAGTCTTCTTTTTTTTTTTCACAGGACGCAGGCTTC
ATCGGCTCCTGGCATCGCCATGATCAGCTCATCTTCCTCGAGCTCGAGCC
TCCCCAGCGGGACGTCGCCGAGCCGCAGGCGTTCTTCCGCCGCACCAACA
GCGCCATCCGCCGCCTGCAGGCCGCCGAAACCTTGGAAAGCGCCTGCGCC
GCCGCGGCGCAAGAGGTGCGGAAGATTACCGGCTTCGATCGGGTGATGAT
CTATCGCTTCGCCTCCGACTTCAGCGGCGAAGTGATCGCAGAGGATCGGT
GCGCCGAGGTCGAGTCAAAACTAGGCCTGCACTATCCTGCCTCAACCGTG
CCGGCGCAGGCCCGTCGGCTCTATACCATCAACCCGGTACGGATCATTCC
CGATATCAATTATCGGCCGGTGCCGGTCACCCCAGACCTCAATCCGGTCA
CCGGGCGGCCGATTGATCTTAGCTTCGCCATCCTGCGCAGCGTCTCGCCC
GTCCATCTGGAATTCATGCGCAACATAGGCATGCACGGCACGATGTCGAT
CTCGATTTTTCGCGGCGAGCGACTGTGGGGATTGATCCTTTTGCCATCAC
CGAACGCCGTACTACGTTGATCTCGATGGCCGCCAAGCCTGCGAGCTAGT
CGCCCAGGTTCTGGCCTGGCAGATCGGCGTGATGGAAGAGTGA
CRa-U6: variant 1 nucleotide sequence
(SEQ ID NO: 12)
GGGTAGTTTGCAGTTTTAAAATTATGTTTTAAAATGGACTATCATATGCT
TACCGTAACTTGAAATATAGAACCGATCCTCCCATTGGTATATATCCAAT
GGGAGGATCGGTTCTATACTTGTGGAAAGGACGAAACACCG
CRa-U6: variant 1 protein sequence
(SEQ ID NO: 13)
GFAVEKLCFKMDYHMETVINIEPILPLVYIQWEDREYTCGKDETP
CRa-U6: variant 2 nucleotide sequence
(SEQ ID NO: 14)
GGGTAGTTTGCAGTTTTAAAATTATGTTTTAAAATGGACTATCATATGCT
TACCGTAACTTGAAACCAATGGGAGGATCGGTTCTATATATATATTATAG
AACCGATCCTCCCATTGGCTTGTGGAAAGGACGAAACACCG
CRa-U6: variant 2 protein sequence
(SEQ ID NO: 15)
GFAVEKLCFKMDYHMLINTNQWEDRFYIYTIEPILPLACGKDETP
CRa-U6: variant 3 nucleotide sequence
(SEQ ID NO: 16)
GGGTAGTTTGCAGTTTTAAAATTATGTTTTAAAATGGACTATCATATGCT
TACCGTAACTTGAAATATAGAACCGATCCTCCCATTGGTATATATTATAG
AACCGATCCTCCCATTGGCTTGTGGAAAGGACGAAACACCG
CRa-U6: variant 3 protein sequence
(SEQ ID NO: 17)
GFAVLKLCFKMDYHMLTVTNIEPILPLVYIIEPILPLACGKDETP
CrA-U6: alternative design
(SEQ ID NO: 18)
AAGGTCGGGCAGGAAGAGGGCCTATTTCCCATGATFCGTTCATATTTGCA
TATACGATAGAAGGCTGTTAGAGAGATAATTAGAATTAATTTGACTGTAA
ACACAAAGATATTAGTACAAAATACGTGACGTAGAAAGTAATAATTTCTT
GGGTAGTTTGCAGTTTTAAAATTATGTTTTAAAATGGACTATCATATGCT
TACCGTAACTTGAAATATAGAACCGATCCTCCCATTGGGTATTTCGATTT
CTTGGCTTTATATATTATAGAACCGATCCTCCCATTGGCTTGTGGAAAGG
AGCAAACACCG
CrA-U6: alternative design
(SEQ ID NO: 19)
KVGQEEGLFPMIPSYLHIRYKAVREIIRINTVNTKILVQNTRRKFLGFAV
LKLCFKMDYHMLTVTNIEPILPLGISISWLYILNRSSHWLVERTKH
gRNA-a target sequence at hybrid promoter
(SEQ ID NO: 20)
TATAGAACCGATCCTCCCATTGG
gRNA-b target sequence at hybrid promoter
(SEQ ID NO: 21)
TACCTCATCAGGAACATGTTGG
gRNA-a regulated by an RNA Pol II promoter
(SEQ ID NO: 22)
TTTCTCGAGTTTACTCCCTATCAGTGATAGAGAACGTATGTCGAGTTTAC
TCCCTATCAGTGATAGAGAACGATTCGAGTTTACTCCCTATCAGTGATAG
AGAACGTATGTCGAGTTTACTCCCTATCAGTGATAGAGAACGTATGTCGA
GTTTACTCCCTATCAGTGATAGAGAACGTATGTCGAGTTTATCCCTATCA
GTGATAGAGAACGTATGTCGAGTTTACTCCCTATCAGTGATAGAGAACGT
ATGTCGAGGTAGGCGTGTACGGTGGGAGGCCTATATAAGCAGAGCTCGTT
TAGTGAACCGTCAGATCGCCTATAGAACCGATCCTCCCATGTTTTAGAGC
TAGAAATAGCAAGTTAAAATAAGGCTAGTCCGTTATCAACTTGAAAAAGT
GGCACCGAGTCGGTGCCTAGTAATAAAGGATCCTTTATCTTCATTGGATC
CGTGTGTTGGTTTTTTGTGTGCGGCCCGTCTAG
gRNA intron of mKate fluorescent protein coding
gene
(SEQ ID NO: 23)
GTTTTAGAGCTAGAAATAGCAAGTTAAAATAAGGCTAGTCCGTTATCAAC
TTGAAAAAGTGGCACCGAGTCGGTGC
igRNA-L1 target sequence at hybrid promoter
(with [symbol] )
(SEQ ID NO: 24)
ACGTCAACGTTTCGCACCATCGG
igRNA-L2 target sequence at hybrid promoter
(with [symbol] )
(SEQ ID NO: 25)
GCTTAATACGGGCTAATCTTGGG
igRNA-L3 target sequence at hybrid promoter
(with [symbol] )
(SEQ ID NO: 26)
ACTTGGCTACCTCGTTCGACAGG
igRNA-L4 target sequence at hybrid promoter
(with [symbol] )
(SEQ ID NO: 27)
TTGGCCTACGTACTGCTCTATGG
igRNA-L5 target sequence at hybrid promoter
(with [symbol] )
(SEQ ID NO: 28)
ACTAGCTATAGATTATCCTAGGG
igRNA-L1 target sequence at hybrid promoter
(SEQ ID NO: 29)
ACGTCAACGTTTCGCACCAT
igRNA-L2 target sequence at hybrid promoter
(SEQ ID NO: 30)
GCTTAATACGGGCTAATCTT
igRNA-L3 target sequence at hybrid promoter
(SEQ ID NO: 31)
ACTTGGCTACCTCGITCGAC
igRNi4-L4 target sequence at hybrid promoter
(SEQ ID NO: 32)
TTGGCCTACGTACTGCTCTA
igRNA-L5 target sequence at hybrid promoter
(SEQ ID NO: 33)
ACTAGCTATAGATTATCCTA

1. E. Andrianantoandro, S. Basu, D. K. Karig et al., Molecular systems biology 2, 2006 0028 (2006).
2. W. C. Ruder, T. Lu, and J. J. Collins, Science 333 (6047), 1248 (2011).
3. A. L. Slusarczyk, A. Lin, and R. Weiss, Nature reviews. Genetics 13 (6), 406 (2012).
4. John Bird, Newnes p. 532. ISBN 978-0-7506-8555-9 (2007).
5. C. S Peirce, Collected Papers v. 4, paragraphs 12-20 (1933).
6. F. Farzadfard, S. D. Perli, and T. K. Lu, ACS synthetic biology (2013).
7. A. S. Khalil, T. K. Lu, C. J. Bashor et al., Cell 150 (3), 647 (2012).
8. A. Garg, J. J. Lohmueller, P. A. Silver et al., Nucleic acids research 40 (15), 7584 (2012).
9. B. P. Kramer, C. Fischer, and M. Fussenegger, Biotechnology and bioengineering 87 (4), 478 (2004).
10. B. C. Stanton, A. A. Nielsen, A. Tamsir et al., Nature chemical biology 10 (2), 99 (2014).
11. Y. Fu, J. A. Foden, C. Khayter et al., Nature biotechnology 31 (9), 822 (2013).
12. P. Mali, L. Yang, K. M. Esvelt et al., Science 339 (6121), 823 (2013).
13. L. S. Qi, M. H. Larson, L. A. Gilbert et al., Cell 152 (5), 1173 (2013).
14. A. A. Nielsen, T. H. Segall-Shapiro, and C. A. Voigt, Current opinion in chemical biology 17 (6), 878 (2013).
15. J. R. Henriksen, C. Lokke, M. Hammero et al., Nucleic acids research 35 (9), e67 (2007).
16. J. K. Ko, K. H. Choi, X. Zhao et al., FASEB journal: official publication of the Federation of American Societies for Experimental Biology 25 (8), 2638 (2011).
17. H. Xia, Q. Mao, H. L. Paulson et al., Nature biotechnology 20 (10), 1006 (2002).
18. J. C. Giering, D. Grimm, T. A. Storm et al., Molecular therapy: the journal of the American Society of Gene Therapy 16 (9), 1630 (2008).
19. S. L. Lin, D. Chang, D. Y. Wu et al., Biochemical and biophysical research communications 310 (3), 754 (2003).
20. S. Cardinale and A. P. Arkin, Biotechnology journal 7 (7), 856 (2012).
21. Domitilla Del Vecchio Andras Gyorgy, PLOS Computational Biology-Accepted 2014.
22. P. P. Dennis, M. Ehrenberg, and H. Bremer, Microbiology and molecular biology reviews: MMBR 68 (4), 639 (2004).
23. S. Klumpp and T. Hwa, Proceedings of the National Academy of Sciences of the United States of America 105 (51), 20245 (2008).
24. K. M. Esvelt, P. Mali, J. L. Braff et al., Nature methods 10 (11), 1116 (2013).
25. J.; Weiss Beal, R.; Yaman F.; Davidsohn N.; Adler A, MIT CSAIL Tech Report 2012-008 (2012).

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.” The term “or” is generally employed in its sense including “and/or” unless the content clearly dictates otherwise.

It should be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended (including but not limited to). Only the transitional phrases “consisting of” and “consisting essentially of” shall be closed or semi-closed transitional phrases, respectively, as set forth in the United States Patent Office Manual of Patent Examining Procedures, Section 2111.03. It should also be understood that all open-ended transitional phrases may be substituted with closed or semi-closed transitional phrases. Thus, the term “comprising” may be substituted with “consisting of” or “consisting essentially of.”

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

1. An engineered nucleic acid comprising
a CRISPR-responsive promoter (CRP) comprising a transcription start site flanked by a first gRNA target site and a second gRNA target site.

2. The engineered nucleic acid of claim 1, wherein the promoter is operably linked to a nucleic acid encoding a product.

3. The engineered nucleic acid of claim 1, wherein the promoter is a RNA Pol II promoter.

4. The engineered nucleic acid of claim 1, wherein the promoter is a RNA Pol III promoter.

5. The engineered nucleic acid of claim 1, wherein the product is a detectable molecule.

6. The engineered nucleic acid of claim 1, wherein the product is a guide RNA (gRNA).

7. The engineered nucleic acid of claim 1, further comprising a response element located upstream from the first gRNA target site.

8. An engineered nucleic acid comprising
a RNA Pol II promoter flanked by a first gRNA target site and a second gRNA target site, wherein the RNA Pol II promoter is operably linked to a nucleic acid encoding a product.

9-13. (canceled)

14. An engineered nucleic acid comprising
a RNA Pol III promoter comprising a sense strand, an antisense strand and a TATA sequence flanked by a first gRNA target site and a second gRNA target site.

15-20. (canceled)

21. A cell comprising the engineered nucleic acid of claim 1.

22. The cell of claim 21 further comprising an engineered nucleic acid comprising a promoter operably linked to a nucleic acid encoding a gRNA that binds to the first and second gRNA target sites of the engineered nucleic acid.

23. The cell of claim 22, wherein the nucleic acid encoding the gRNA is flanked by cognate intronic splice sites and is located within a nucleic acid encoding a detectable molecule.

24. The cell of claim 22, wherein the nucleic acid encodes multiple gRNAs.

25. The cell of claim 22, wherein the engineered nucleic acid comprises a response element upstream of the promoter operably linked to a nucleic acid encoding a gRNA.

26. (canceled)

27. The cell of claim 21 further comprising Cas9.

28. (canceled)

29. The cell of claim 27, wherein the Cas9 is catalytically inactive Cas9m.

30. A cell comprising at least two engineered nucleic acids of claim 1, wherein
(a) the promoter of one of the engineered nucleic acids is operably linked to a nucleic acid encoding a gRNA, and
(b) the promoter of another of the engineered nucleic acids is operably linked to a nucleic acid encoding a detectable molecule.

31. (canceled)

32. The cell of claim 30, wherein the gRNA of (a) binds to the first and second gRNA target sites of the engineered nucleic of (b).

33. (canceled)

34. A library comprising pairs of engineered nucleic acids, wherein each pair comprises:
(a) the engineered nucleic acid of claim 1;
(b) an engineered nucleic acid comprising a nucleic acid encoding a gRNA, wherein the gRNA binds to the first and second gRNA target sites of the engineered nucleic acid of (a).

35-39. (canceled)

40. The cell of claim 1, further comprising:
(a) a response element located upstream from the CRISPR-responsive promoter comprising a transcription start site flanked by a first gRNA target site and a second gRNA target site, wherein the promoter is operably linked to a detectable molecule;
(b) an engineered nucleic acid comprising a CRISPR-responsive promoter comprising a transcription start site flanked by a third gRNA target site and a fourth gRNA target site, wherein the promoter is operably linked to a nucleic acid encoding a gRNA that binds to the first and second gRNA target sites of the engineered nucleic acid of (a); and
(c) an engineered nucleic acid comprising a promoter operably linked to a nucleic acid encoding a gRNA that binds to the third and fourth gRNA target sites of the engineered nucleic acid of (b).

41-51. (canceled)
